(19) United States
(12) Patent Application Publication
(10) Pub. No.: US 2021/0369911 A1
(43) Pub. Date: Dec. 2, 2021

(54) HYDROGEL COMPOSITIONS

(71) Applicants: OPHTALMIC COMPAGNIE, VILLEPINTE (FR); Université de Haute-Alsace, MULHOUSE (FR)

(72) Inventors: Jean-François STUMBÉ, STRASBOURG (FR); Fanny COUMES, LEUC (FR); Jean-François RUMIGNY, SURZUR (FR); Sophie BISTAC, GALFINGUE (FR); Romain JAGU, DREFFÉAC (FR)

(21) Appl. No.: 17/282,121

(22) PCT Filed: Sep. 30, 2019
(86) PCT No.: PCT/FR2019/052302

§ 371 Date: Apr. 1, 2021

(30) Foreign Application Priority Data
Oct. 2, 2018 (FR) 1859124

(51) Int. Cl. A61L 27/18; A61K 9/06; A61K 47/34; A61K 47/10; A61L 27/52

(52) U.S. Cl. CPC A61L 27/18; A61K 9/06; A61L 2430/16; A61K 47/10; A61L 27/52; A61K 47/34

(57) ABSTRACT

A crosslinkable composition comprises: A1) at least one multifunctional isocyanate-terminated urethane prepolymer comprising between 1 and 16 isocyanate functionalities on average, the prepolymer being a product of the reaction of a diisocyanate, a triisocyanate or a polyisocyanate with a functionality strictly greater than 3, with a polyol comprising 1 to 8 hydroxyl groups; and/or A2) at least one mono, di or polyisocyanate and/or an oligoglycerol; with B) at least one macropolyol chosen from: B1) oligoglycerols with an average degree of polymerisation less than or equal to 7, B2) glycerol dendrimers, B3) linear, branched or hyperbranched polyglycerols with a degree of polymerisation greater than or equal to 8, B4) mixtures of hyperbranched polyglycerols and linear, branched or hyperbranched oligoglycerols, with a degree of polymerisation of between 2 and 7, optionally functionalised.

### TECHNICAL FIELD

The present invention relates to crosslinkable and crosslinked compositions, as well as to a hydrogel obtainable from the crosslinked composition.

The present invention finds industrial applications in the field of biocompatible materials, and in particular that of ocular lenses.

In the description below, references in square brackets ([ ]) refer to the list of references at the end of the text.

### STATE OF THE ART

A contact lens, worn on the eye, is used to correct vision defects such as myopia, astigmatism, or hyperopia. However, the human eye needs to maintain a certain level of hydration and oxygen circulation. Thus, the lens in contact with the eye must meet a set of specifications that include—but are not limited to—good oxygen permeability, good comfort, and hydrophilicity.

Contact lenses can be classified into two categories: rigid contact lenses, including rigid gas permeable lenses, and soft contact lenses, such as hydrogel or silicone hydrogel lenses.

During the production of polymer-based contact lenses, a polymerizable lens precursor composition is polymerized to form a crosslinked contact lens product, which can then be processed to form a hydrated contact lens. For example, the polymerizable precursor composition may be placed on a cavity of a contact lens-shaped mold, and may be polymerized therein to form a contact lens located in the cavity. Polymerization can be achieved by exposing the polymerizable composition by heating in the optional presence of thermal initiator or by exposure to ultraviolet light.

A hydrogel is a hydrated crosslinked polymer system that contains water in an equilibrium state. It is typically oxygen permeable and biocompatible, which makes it a preferred material for producing biomedical devices and in particular contact or intraocular lenses. Hydrogel soft lenses are manufactured from few basic monomers. Their choice will depend on the final properties that the lens manufacturer wishes to favor:
better oxygen permeability increasing with water content or with the use of siliconized and/or fluorinated co-monomers and/or macromonomers,
better resistance to deposits, for example lipid, protein and bacterial deposits,
better resistance to dehydration, which tends to increase with the water content, but also and above all with the retention capacity of the polymer used, which depends on the chemical composition.

Often, conventional hydrogel contact lenses are the polymerized product of a composition of lens precursor(s) containing hydrophilic monomers such as 2-hydroxyethyl methacrylate (HEMA), methacrylic acid (MAA), methyl methacrylate (MMA), N-vinylpyrrolidone (NVP), but also optionally additives of polymeric nature such as polyvinyl alcohol (PVA), polyvinylpyrrolidone (PVP) and combinations thereof. The precursor compositions also frequently contain one or more catalysts and one or more crosslinking agents.

Although these lenses provided some comfort, they did not provide sufficient oxygen permeability to prevent the problems associated with corneal hypoxia. Attempts to resolve this problem included copolymerizing HEMA with hydrophilic monomers such as methacrylic acid and N-vinylpyrrolidone. Although these polymers increase the level of oxygen permeability, the incorporation of these comonomers also leads to problems such as protein and lipid deposition, corneal dryness, and lens dehydration.

More recently, a new generation of polymers has been developed to further increase the oxygen level. These materials are based on the copolymerization of silicone or silane functionalized methacrylate co-monomers with hydrophilic co-monomers. Lenses produced from these materials were initially designed for extended use. Although they have succeeded in increasing oxygen permeability, these new materials still suffer from limitations such as lipid deposition and dry eye, which reduces eye comfort.

Moreover, current contact lenses made of silicone or PHEMA hydrogels are often associated with poor biocompatibility, as they trigger an immune response when biomolecules such as proteins, lipids, immunoglobulins and complement proteins bind to the lens surface. This reduces the stability of the tear film, causing the eye to feel dry.

One class of polymers for new contact lens materials is that of polyethylene glycol (PEG)-based polyurethanes, as described for example in the document EP2496620 ([1]), which illustrates a material comprising PEG diol, polyol, diisocyanate and polydimethylsiloxane (PDMS) diol. However, no lenses comprising this class of polymer are yet commercially available. There is thus a genuine need for new materials that overcome these defects, disadvantages and obstacles of the prior art, in particular having particularly attractive properties in terms of oxygen permeability, water content, resistance to water loss or retention, mechanical properties and strength (Young's modulus, stress and elongation at break), but also in terms of surface properties such as the coefficient of friction, surface roughness and wettability.

### DESCRIPTION OF THE INVENTION

After substantial research, the Applicant has succeeded in developing a new material that meets the above-mentioned needs and is therefore particularly suitable for use in the manufacture of contact lenses.

Surprisingly, the Applicant has succeeded in creating a transparent hydrogel capable of swelling in an aqueous medium, and showing very attractive properties compared with existing materials, in particular by using polyglycerol, in particular hyperbranched polyglycerol, combined advantageously with other polyols and polyisocyanates.

Advantageously, the medical device is biocompatible, and in particular suitable for contact with the eyes, and hydrophilic. Furthermore, it has particularly attractive properties in terms of oxygen permeability, water content, resistance to water loss or retention, mechanical properties and resistance (Young's modulus, stress and elongation at break) but also in terms of surface properties such as the coefficient of friction, surface roughness and wettability.

Surprisingly, the Applicant has succeeded in synthesizing a hydrogel by reacting a macropolyol, in particular a hyperbranched polyglycerol, with a polyisocyanate, in particular a diisocyanate.

In particular, the Applicant has succeeded, in one aspect of the invention, in synthesizing a hydrogel by reacting a macropolyol, in particular a hyperbranched polyglycerol, with an isocyanate-terminated prepolymer and/or by direct reaction with a mixture of polyols and polyisocyanates.

Thus, a first object of the invention relates to a crosslinkable composition comprising:

A1) at least one multifunctional isocyanate-terminated urethane prepolymer comprising from 1 to 16 isocyanate functionalities on average, the average functionality being strictly greater than 1, the prepolymer being a product of the reaction of a diisocyanate, a triisocyanate, or a polyisocyanate of functionality strictly greater than 3, preferably aliphatic or cycloaliphatic and liquid at 25° C.±3° C., with a monofunctional polyol or alcohol comprising 1 to 8, preferably 2 to 3, hydroxyl groups; and/or
A2) at least one mono-, di- or polyisocyanate and/or oligoglycerol, and
B) at least one macropolyol selected from:
B1) oligoglycerols with an average DP of 7 or less,
B2) glycerol dendrimers,
B3) linear, branched or hyperbranched polyglycerols with an average degree of polymerization greater than or equal to 8,
B4) mixtures of hyperbranched polyglycerols and linear, branched or hyperbranched oligoglycerols, with an average degree of polymerization comprised between 2 and 7, optionally functionalized;
   C) optionally at least one polyol comprising at least two hydroxyl groups;
   D) optionally at least one mono-, di- or polyisocyanate;
   E) optionally at least one monoalcohol, in particular a linear or branched, saturated or unsaturated, cyclic or acyclic monoalcohol having from 1 to 30 carbon atoms, a mixture of monofunctional alcohols or a monohydroxylated polyether based on ethylene glycol and/or ethylene glycol/propylene glycol, or a mixture of monohydroxylated polyether and monofunctional alcohols; the monohydroxy polyether can for example have a molar mass comprised between 300 g/mol and 5000 g/mol;
   F) optionally at least one catalyst or combination of catalysts;
   G) optionally at least one additive selected from antioxidants, oxygen permeability promoters, water retention agents, corneal lubricating agents, compatibilizing agents, coloring agents, opacifying agents, antimicrobial agents, viscosity modifying agents, therapeutic agents and bacterial anti-biofilm agents; and/or
   H) optionally at least one agent selected from UV filters, UV absorbers and blue-light filters.

A second object of the invention relates to a crosslinked composition capable of forming a hydrogel polymer by absorption of water, resulting from the crosslinking of a crosslinkable composition of the invention, either under anhydrous conditions or in the presence of small amounts of water, in particular so as to promote the formation of urea bonds, and either under an inert atmosphere or under an ambient atmosphere, for example according to a process for preparing a crosslinked composition according to the invention as described below.

“Crosslinkable composition”, in the sense of the present invention, means a composition which is liquid at room temperature and which can be converted to a material, in particular by reaction of its various constituents after bringing into contact by mixing, optionally adding catalysts and increasing the temperature.

The crosslinkable composition has the following characteristics:

“Prepolymer”, in the sense of the present invention, means an oligomer or a polymer having reactive groups which enable it to participate in a subsequent polymerization, and thus to incorporate several monomer units into at least one chain of the final macromolecule or into the final polymeric material constituting the hydrogel.

The prepolymer may be liquid at room temperature (i.e., about 25° C.±3° C.), or it may be solid at room temperature. The prepolymer is preferably liquid at the temperature at which the formation reaction is carried out.

The prepolymer of the present invention may be a multifunctional isocyanate-terminated urethane prepolymer comprising from 1 to 16 isocyanate functionalities. For example, it may be 0, 1, 2, or 3, or 4, or 5, or 6, or 7, or 8, or 9, or 10, or 11, or 12, or 13, or 14, or 15, or 16 isocyanate functionalities. The average functionality is strictly greater than 1 for crosslinking and hydrogel network formation to occur. For example, the average functionality may be at least 2, or at least 3, or at least 4, or at least 5, or at least 6, or at least 7, or even greater than 7. Advantageously, in a mixture of isocyanates and polyisocyanates as described in the invention, molecules of functionality 2 must be present. The average functionality can be calculated as the molar average of the functionalities of the individual molecules.

Advantageously, the prepolymer can be a crosslinking agent.

The prepolymer is prepared separately, before mixing with the other components of the crosslinkable composition of the invention, by pre-reacting a polyisocyanate, for example a diisocyanate, triisocyanate or polyisocyanate of functionality strictly greater than 3, with a polyol, which may be linear or branched, comprising 1 to 8, preferably 2 to 6, hydroxyl groups.

The diisocyanate, triisocyanate and polyisocyanate with a functionality strictly greater than 3, hereinafter referred to as “polyisocyanate(s)”, may be aliphatic or cycloaliphatic. They are liquid at room temperature (about 25° C.±3° C.).

The triisocyanate can be, for example, aliphatic and without aromatic units. It can be, for example, functional trimer (isocyanurate) of isophorone diisocyanate, hexamethylene diisocyanate trimer.

The prepolymer can be derived from a polyisocyanate with a functionality greater than 2, for example an isocyanurate or an allophanate.

Whether used in the context of the prepolymer or as a constituent as such of the crosslinked and/or crosslinkable composition, the diisocyanate may be of the following formula: OCN—R₁—NCO, wherein R₁ represents a linear or branched, monocyclic or polycyclic or acyclic C₁ to C₁₅ alkylene, a C₁ to C₄ alkylidene, or a C₆-C₁₀ arylene optionally bearing at least one substituent selected from linear or branched C₁-C₆ alkyl, C₁-C₂ alkylene, or halogen atom. The diisocyanate may be selected from methylene dicyclohexyl diisocyanate, hexamethylene diisocyanate, isophorone diisocyanate, toluene-2,4-diisocyanate, toluene-2,6-diisocyanate, mixtures of toluene-2,4 and 2,6-diisocyanates, ethylene diisocyanate, ethylidene diisocyanate, propylene-1,2-diisocyanate, cyclohexylene-1,2-diisocyanate, cyclohexylene-1,4-diisocyanate, m-phenylene diisocyanate, 4,4′-biphenylene diisocyanate, 3,3′-dichloro-4,4′-biphenylene diisocyanate, 1,4-tetramethylene diisocyanate, 1,10-decamethylene diisocyanate, cumene-2,4 diisocyanate, 1,5-naphthalene diisocyanate, 1,4-cyclohexylene diisocyanate, 2,5-fluorenediisocyanate, 2,2′-diphenylmethylene diisocyanate, 4,4′-diphenylmethylene diisocyanate, 4,4′-dibenzyl diisocyanate, m-xylylene diisocyanate, hexamethylene diisocyanate trimer, and polymers of 4,4′-diphenylmethane diisocyanate, diphenyl-4,4″-biphenylene diisocyanate, 1,6-hexamethylene diisocyanate, m-phenylene diisocyanate, p-tetramethyl xylylene diisocyanate, p-phenylene diisocyanate, 4-methoxy-1,3-phenylene diisocyanate, 4-chloro-1,3-phenylene diisocyanate, 4-bromo-1,3-phenylene diisocyanate, 4-ethoxy-1,3-phenylene diisocyanate, 2,4-dimethyl-1,3-phenylene diisocyanate, 5,6-dimethyl-1,3-phenylene diisocyanate, 2,4-diisocyanatodiphenylether, 4,4″-diisocyanatodiphenylether, benzidine diisocyanate, 4,6-dimethyl-1,3-phenylene diisocyanate, 1,4-anthracene diisocyanate, 4,4′-diisocyanatodibenzyl, 3,3′-dimethyl-4,4′-diisocyanatodiphenylmethane, 2,6-dimethyl-4,4′-diisocyanatodibenzyl, 2,4-diisocyanatostilbene, 3,3′-dimethoxy-4,4′-diisocyanatodiphenyl, 2,5-fluorenediisocyanate, 1,8-naphthalene diisocyanate, 2,6-diisocyanatobenzofuran, xylene diisocyanate, m-tetramethyl xylylene diisocyanate or methylene diisocyanatodiphenyl. Preferably, it can be hexamethylene diisocyanate, isophorone diisocyanate or methylenebis(4-cyclohexyl)diisocyanate.

The polyol used in the context of the preparation of the prepolymer, also called polyalcohol monofunctional alcohol or glycol, can comprise from 1 to 8 hydroxyl groups, for example 1, or 2, or 3, or 4, or 5, or 6, or 7, or 8 hydroxyl groups, preferably 2 to 3 hydroxyl groups. It can be selected from linear or branched poly(ethylene glycols) (PEG) or poly(propylene glycols) (PPG) comprising at least one hydroxyl function; molecular polyols comprising at least one hydroxyl function; co-polymers of poly(ethylene glycols) (PEG) and poly(propylene glycols) (PPG); and diols comprising silanes or polysiloxanes with terminal hydroxyl functions. The polyol may be particularly selected from ethylene glycol, diethylene glycol, triethylene glycol, trimethylolpropane, glycerol, pentaerythritol, xylitol, sorbitol, ethanol, butanol, phenol, 1,2-propylene glycol, dipropylene glycol, 1,4-butane diol, hexamethylene glycol, polydimethylsiloxanediols, and linear or branched PEG/PPG copolymers, such as the compounds in the Pluronic® or Tetronic® commercial ranges.

Advantageously, the PEGdiols and PPGdiols can have a molar mass comprised between 200 g/mol and 6000 g/mol, preferentially between 200 g/mol and 5000 g/mol. For example, the prepolymer obtained may be poly(ethylene glycol)-hexamethylene diisocyanate (i.e., a difunctional polyethylene glycol functionalized at each end with a hexamethylene diisocyanate, leading to a polyethylene glycol with two terminal isocyanate functions), poly(ethylene glycol)-isophorone diisocyanate (i.e., a difunctional polyethylene glycol functionalized at each end with isophorone diisocyanate, leading to a polyethylene glycol with two terminal isocyanate functions), methylenebis(4-cyclohexyl)isocyanate poly(ethylene glycol) diisocyanate (PEG-(h-MDI)₂) (i.e., a difunctional polyethylene glycol functionalized at each end with methylenebis(4-cyclohexyl)isocyanate, leading to a polyethylene glycol with two terminal isocyanate functions), or trimethylene propane ethoxylate-triisocyanate hexamethylene diisocyanate (i.e., a trifunctional polyethylene glycol functionalized at each end with hexamethylene diisocyanate, leading to a polyethylene glycol with three terminal isocyanate functions). The reaction between the diisocyanate or triisocyanate and the polyol, allowing the formation of the prepolymer, can take place in a solvent medium, for example an aprotic polar solvent of the dimethylformamide, acetonitrile or dimethylsulfoxide type. Alternatively, the reaction can take place in bulk, i.e., without solvent. In solventless systems, the mixture between the prepolymer and the polyisocyanate is liquid at the processing temperature. For example, the prepolymer can be prepared at temperatures of about 60° C. in solventless systems, if necessary.

In the context of the preparation of the prepolymer, the isocyanate/hydroxyl stoichiometric ratios used can be between 2.2:1 and 1.1:1, for example 2:1, particularly in the case of reactions between diisocyanates and diols. The reaction temperatures can range from room temperature to 120° C., preferably between 25 and 80° C., more preferentially at 70° C., for example for 1 to 12 h until the prepolymer is formed, which can be characterized by ¹H NMR, IR, SEC or by titration of the residual NCO groups.

In the context of the preparation of the prepolymer, the addition of a catalyst is optional, but may be carried out to accelerate the reaction rate. The catalyst may be selected from organobismuth, organometallic tin salts such as tin dibutyl laurate and/or tin octoate, iron tetrachloride, tertiary amines such as triethylamine, and mixtures thereof. The catalyst may be present in an amount of 0.01 to 2% by weight of the reactants, for example from 0.03 to 0.08% by weight of the prepolymer, preferably 0.05% by weight.

In the context of the preparation of the prepolymer, prior to the reaction with the polyisocyanate, the polyol can be dried under reduced pressure at a temperature comprised between 60 and 100° C., preferably between 70 and 90° C., for 12 to 24 hours to avoid the presence of moisture; indeed, the prepolymer formed is reactive in the presence of water, so it is preferable to dry the multifunctional compounds and the polyols/diols before proceeding with the mixing. It can then be introduced as is into the reaction medium if the reaction takes place in bulk, or as a solution if the reaction takes place in solvent under inert atmosphere (argon/nitrogen). The polyisocyanate used can be taken under an inert gas (nitrogen/argon) flow and added as is to the reaction medium if the reaction takes place in bulk, or as a solution if the reaction takes place in solvent, for example under stirring between 25° C. (ambient temperature) and 120° C., advantageously between 40 and 90° C., such as for example at 70° C. The reaction product can be recovered hot under inert gas (argon/nitrogen) flow and stored cold, for example at temperatures close to zero, or down to 5° C.

When at least one monoisocyanate is used as a constituent of the composition as such, apart from that incorporated in the prepolymer, it can advantageously have a chain-limiting role for modulus modulation. This can be any monoisocyanate suitable for the reaction conditions, in particular liquid aliphatic or aromatic isocyanates, such as butyl isocyanate.

When at least one diisocyanate is used as a constituent of the composition as such, apart from that incorporated in the prepolymer, it may be aromatic, aliphatic or cycloaliphatic, preferably aliphatic or cycloaliphatic. The diisocyanate may have the following formula: OCN—R₁—NCO, wherein R₁ is a linear or branched, monocyclic or polycyclic or acyclic C₁ to C₁₅ alkylene, a C₁ to C₄ alkylidene, or a C₆-C₁₀ arylene optionally bearing at least one substituent selected from linear or branched C₁-C₆ alkyl, C₁-C₂ alkylene, or a halogen atom. When a polyisocyanate is used as a constituent as such, it may have an average functionality greater than or equal to 2, for example 3 or 4. Preferably, the di- or polyisocyanate is aliphatic and free of aromatic units, and liquid at room temperature (i.e., about 25° C.). The di- or polyisocyanate is used in an amount between 1 and 60% by weight, for example between 5 and 50% by weight. In any event, the amount of di- or polyisocyanate can be adjusted, according to the general knowledge of the person skilled in the art, and in light of the present invention, to modify the properties of the hydrogel of the invention. The di- and polyisocyanates may be selected from methylene dicyclohexyl diisocyanate, hexamethylene diisocyanate, isophorone diisocyanate, toluene-2,4-diisocyanate, toluene-2,6-diisocyanate, mixtures of toluene 2,4 and 2,6-diisocyanates, ethylene diisocyanate, ethylidene diisocyanate, propylene-1,2-diisocyanate, cyclohexylene-1,2-diisocyanate, cyclohexylene-1,4-diisocyanate, m-phenylene diisocyanate, 4,4′-biphenylene diisocyanate, 3,3′-dichloro-4,4′-biphenylene diisocyanate, 1,4-tetramethylene diisocyanate, 1,10-decamethylene diisocyanate, cumene-2,4 diisocyanate, 1,5-naphthalene diisocyanate, 1,4-cyclohexylene diisocyanate, 2,5-fluorenediisocyanate, 2,2′-diphenylmethylene diisocyanate, 4,4′-diphenylmethylene diisocyanate, 4,4′-dibenzyl diisocyanate, m-xylylene diisocyanate, hexamethylene diisocyanate trimer, methylene dicyclohexyl diisocyanate, biphenylene diisocyanate, 1,6-hexamethylene diisocyanate, m-phenylene diisocyanate, 1,4-tetramethylene diisocyanate, polymers of 4,4′-diphenylmethane diisocyanate, p-tetramethyl xylylene diisocyanate, p-phenylene diisocyanate, 4-methoxy-1,3-phenylene diisocyanate, 4-chloro-1,3-phenylene diisocyanate, 4-bromo-1,3-phenylene diisocyanate, 4-ethoxy-1,3-phenylene diisocyanate, 2,4-dimethyl-1,3-phenylene diisocyanate, 5,6-dimethyl-1,3-phenylene diisocyanate, 2,4-diisocyanatodiphenylether, 4,4″-diisocyanatodiphenylether, benzidine diisocyanate, 4,6-dimethyl-1,3-phenylene diisocyanate, 1,4-anthracene diisocyanate, 4,4′-diisocyanatodibenzyl diisocyanate, 3,3′-dimethyl-4,4′-diisocyanatodiphenylmethane, 2,6-dimethyl-4,4′-diisocyanatodibenzyl, 2,4-diisocyanatostilbene, 3,3′-dimethoxy-4,4′-diisocyanatodiphenyl, 2,5-fluorenediisocyanate, 1,8-naphthalene diisocyanate, 2,6-diisocyanatobenzofuran, xylene diisocyanate, m-tetramethyl xylylene diisocyanate; preferably hexamethylene diisocyanate, isophorone diisocyanate and methylenebis(4-cyclohexyl)diisocyanate or a polyisocyanate with a functionality greater than 2, for example the trifunctional trimer (isocyanurate) of isophorone diisocyanate or the trifunctional (isocyanurate) trimers of hexamethylene diisocyanate and 4,4-diphenyl methane diisocyanate polymer, as well as the corresponding allophanates, biurets or uretdiones.

Advantageously, the macropolyol used in the invention has a crosslinking role.

“Oligoglycerol”, in the sense of the present invention, means a glycerol polymer, the average degree of polymerization of which is less than or equal to 7, for example from 2 to 7. The degree of polymerization and the average degree of polymerization can be determined by any method known to the person skilled in the art, for example by size-exclusion chromatography and ¹H and ¹³C NMR. The oligoglycerol may be functionalized. The oligoglycerol may be linear, branched or hyperbranched. Oligoglycerols are commercially available, for example diglycerol (INCI Diglycerin), polyglycerol-3 (Polyglycerin-3) and polyglycerol-4 (Polyglycerin-4), distributed by the company Inovyn.

“Glycerol dendrimer”, in the sense of the present invention, means a molecule consisting of 1 or more dendrons emanating from a single constituent unit, a dendron being a molecule having a single free valence or focal unit, comprising exclusively constituent repeating units of a dendritic and terminal nature, wherein each path from the free valence (focal unit) to any of the terminal units comprises the same number of constituent repeating units. The molar mass of a dendrimer may be comprised between 500 and 100 000 g/mol, for example between 500 and 6000 g/mol, or between 800 and 4000 g/mol, having degrees of polymerization (DP) comprised between 5 and 70; dispersities between 1 and 1.8 and degrees of branching (DB) comprised between 0.9 and 1, optionally prepared as described in the paper J. Am. Chem. Soc. 2000, 122, 2954-2955 ([3]).

“Polyglycerol”, in the sense of the invention, means a glycerol polymer consisting of glycerol units linked by ether bonds, the molar mass of which may be between 500 (DP=7) and 100 000 (DP=1350) g/mol (500 may be excluded or included), for example between 500 and 10 000 g/mol, or between 800 and 6000 g/mol. The polyglycerol may have dispersities between 1.1 and 5, for example between 1.1 and 1.8. It can be, for example, linear, branched or hyperbranched polyglycerols. “Linear” oligoglycerol or polyglycerol, in the sense of the present invention, means a glycerol polymer comprising a chain of glycerol units linked together by ether bonds established essentially between the primary alcohol functions of the glycerol.

“Branched” polyglycerol or oligoglycerol, in the sense of the invention, means a glycerol polymer having a branched three-dimensional structure, which may have a DB (Degree of Branching, as defined by Frey, namely DB=(2D/(2D+L)) comprised between 0.05 and 0.3 (Nomenclature and Terminology for Dendrimers with Regular Dendrons and for Hyperbranched Polymers A. Fradet, J. Kahovec, IUPAC Nomenclature Project Nr: 2001-081-1-800 ([2])). In general, the degree of branching (DB) can be determined using classical state of the art methods, for example by inverse-gated ¹³C NMR.

“Hyperbranched” polyglycerol or oligoglycerol, in the sense of the present invention, means a substance composed of hyperbranched macromolecules which consist of chains joined by a common core and a substantial fraction of branched glycerol repeating units and glycerol terminal units but also comprising linear glycerol repeating units such that the degree of branching DB defined by H. Frey is comprised between 0.3 and 0.8, more frequently between 0.3 and 0.7 (A. Fradet and J. Kahovec ([2])).

As mentioned above, the hyperbranched polyglycerol, optionally functionalized, may have a molar mass comprised between 500 and 100 000 g/mol, preferably between 500 and 10 000 g/mol, and may have degrees of polymerization (DP) between 7 and 1350, dispersities between 1.1 and 5, for example between 1.1 and 1.8, and degrees of branching (DB) between 0.3 and 0.7. Polyglycerols with degrees of branching equal to 1 can be used and prepared, for example, according to the method described in the paper J. Am. Chem. Soc. 2000, 122, 2954-2955 ([3]).

Alternatively, the glycerol polymer may be a mixture of hyperbranched polyglycerols and linear, branched or hyperbranched oligoglycerols, for example with a degree of polymerization comprised between 2 and 7, optionally functionalized. In the mixture, the ratio of hyperbranched polyglycerols to linear, branched or hyperbranched oligoglycerols is from 100:0 to 80:20 by weight.

The macropolyol can be obtained by any method known to the skilled person, for example by ring-opening polymerization of glycidol or glycerol carbonate, using one or more mono- or polyfunctional initiators such as, for example, monoalcohols like methanol, butanol, phenol and derivatives thereof, benzyl alcohol, 1-dodecanol, 1-tetradecanol, 1-hexadecanol, glycol monoalkylethers such as glycol monoethyl ethers or polyethylene glycol monoalkylethers, such as for example polyethylene glycol monoethyl ether, diols such as ethylene glycol, diethylene glycol, triethylene glycol, polyethylene glycols, 1,2-propanediol, 1,3-propanediol, 1,2-butanediol, 1,3-butanediol, 2,3-butanediol, 1,2-pentanediol, 1,3-pentanediol, 1,4-pentanediol, 1,5-pentanediol, 2,3-pentanediol, 2,4-pentanediol, 1,2-hexanediol, 1,3-hexanediol, 1,4-hexanediol, 1,5-hexanediol, 1,6-hexanediol, 2,5-hexanediol, 1,2-heptanediol, 1,7-heptanediol, 1,8-octanediol, 1,2-octanediol, 1,9-nonanediol, 1,10-decanediol, 1,2-decanediol, 1,12-dodecanediol, 1,2-dodecanediol, 1,5-hexadiene-3,4-diol, cyclopentanediols, cyclohexanediols, inositol and derivatives thereof, (2)-methyl-2,4-pentanediol, 2,4-dimethyl-2,4-pentanediol, 2-ethyl-1,3-hexanediol, 2,5-dimethyl-2,5-hexanediol, 2,2,4-trimethyl-1,3-pentanediol, pinacol, dipropylene glycol, polypropylene glycols, 1,4-butanediol, hexamethylene glycol, bisphenol A, bisphenol F, bisphenol S, diethylene glycol, triethylene glycol, dipropylene glycol, tripropylene glycol, polyethylene glycols HO(CH₂CH₂O)n-H or polypropylene glycols HO(CH[CH₃]CH₂O)ₙ—H (where n is an integer greater than or equal to 4) or mixtures of two or more of the above compounds. It is also possible to use triols or polyols with a functionality greater than 3 such as glycerol, diglycerol, triglycerol, tetraglycerol, trimethylolethane, trimethylolpropane, di-trimethylolpropane, sorbitol, but also hydroxyl-terminated oligomers such as ethoxylated pentaerythritols and propoxylated pentaerythritols, ethoxylated trimethylolpropanes and propoxylated trimethylolpropanes, ethoxylated or propoxylated glycerols, resulting from the ring-opening addition reaction of ethylene oxide and/or propylene oxide to pentaerythritol, trimethylolpropane and glycerol. The degree of ethoxylation is typically comprised between 0.1 and 10 ethylene oxide units per OH function. The molar mass is generally comprised between 100 and 1000 g/mol. Ethoxylated trimethylolpropanes, ethoxylated glycerols and ethoxylated pentaerythritols may be preferentially used. “Star” molecules with at least three arms comprising polyoxypropylene-polyoxyethylene blocks can also be used such as ethoxylated or propoxylated sorbitols and saccharides, degraded starch, polyvinyl alcohol. It is also possible to use initiators such as water, methylamine, ethylamine, propylamine, butylamine, dodecylamine, myristylamine, palmitylamine, stearylamine, aniline, benzylamine, ortho- or para-toluidine, α,β-naphthylamine, ammonia, ethylene diamine, propylene diamine, 1,4-butylene diamine, 1,2-, 1,3-, 1,4-, 1,5-, or 1,6-hexamethylene diamines, as well as o-, m- and p-phenylene diamines, 2,4- and 2,6-tolylenediamine, 2,2′-, 2,4 and 4,4′-diaminodiphenylmethane, 2,2′-, 2,4 and 4,4′-diaminodicyclohexylmethane, diethylene glycol diamine, diethylene triamine, triethylene tetramine, difunctional or trifunctional poly(propylene glycol)diamines (Jeffamines). Amino alcohols such as diethanolamine, dipropanolamine, diisopropanolamine, triethanolamine, tris(hydroxymethyl)aminomethane or diisopropylethanolamine can also be used, but also compounds containing functional groups such as allyl alcohol, allylglycerol, 10-undecenol, 10-undecenamine, or dibenzylamine.

The initiator used in the context of the preparation of the macropolyol can then be partially deprotonated with a suitable agent selected from alkali metals and their hydrides, alkoxides, hydroxides. Preferentially, metals or metal alkoxides such as potassium methanolate (MeOK) are used. Potassium carbonate can also be used as a catalyst for the polymerization of glycerol carbonate in particular.

Other molecules can be used to catalyze the polymerization to obtain the macropolyol, such as alcoholates, organometallic compounds, metal salts, tertiary amines. Among the alcoholates, alkali metal alcoholates such as sodium methylate, potassium isopropylate or potassium methanolate can be used. It is also possible to use tetraalkylammonium hydroxides, such as tetramethylammonium hydroxides; alkali metal hydroxides, such as sodium hydroxide and potassium hydroxide; metal salts, such as organic and/or inorganic compounds based on iron, lead, bismuth, zinc and/or tin at conventional metal oxidation levels, for example: iron(II) chloride, iron(III) chloride, bismuth(III) 2-ethylhexanoate, bismuth(III) octoate, bismuth(III) neodecanoate, zinc chloride, zinc 2-ethylcaproate, tin(II) octoate, tin(II) ethylcaproate, tin(II) palmitate, tin(IV) dibutyldilaurate (DBTL), tin(IV) dibutyldichloride or lead octaote or amidines, such as 2,3-dimethyl-3,4,5,6-tetrahydropyrimidine. It is also possible to use alkali metal salts of long-chain fatty acids having 10 to 20 carbon atoms and optionally pendant OH groups. It is also possible to use tertiary amines such as triethylamine, tributylamine, dimethylbenzylamine, diethylbenzylamine, pyridine, methylpyridine, dicyclohexylmethylamine, dimethylcyclohexylamine, N,N,N′,N′-tetramethyldiaminodiethyl ether, bis(dimethylaminopropyl)-urea, N-methyl- and N-ethylmorpholine, N-cocomorpholine, N-cyclohexylmorpholine, N,N,N′,N′-tetramethylethylenediamine, N,N,N′,N′-tetramethyl-1,3-butanediamine, N,N,N′,N′-tetramethyl-1,6-hexanediamine, pentamethyldiethylenetriamine, N-methylpiperidine, N-dimethylaminoethylpiperidine, N, N′-dimethylpiperazine, N-methyl-N′-dimethylaminopiperazine, 1,8-diazabicyclo(5.4.0)undec-7-ene (DBU), TBD (1,5,7-triazabicyclo[4.4.0]dec-5-ene), 1,2-dimethylimidazole, 2-methylimidazole, N, N-dimethylimidazole, 3-phenylethylamine, DABCO or 1,4-diazabicyclo-(2,2,2)octane, bis-(N,N-dimethylaminoethyl)adipate; alkanolamine-type compounds, such as triethanolamine, triisopropanolamine, N-methyl- and N-ethyl-diethanolamine, dimethylaminoethanol, 2-(N, N-dimethylaminoethoxy)ethanol, N,N′, N″ tris-(dialkylaminoalkyl)hexahydrotriazines, such as N, N′, N″ tris-(dimethylaminopropyl)-s-hexahydrotriazine and/or (dimethylaminoethyl)ether.

The macropolyol synthesis reaction can take place in the presence of solvent, for example an aliphatic, cycloaliphatic or aromatic solvent such as decalin, toluene or xylene, or an ether such as glyme, diglyme or triglyme. Alternatively, the reaction can take place in bulk, for example between 40 and 140° C., preferably at 95° C. in semi-batch, advantageously corresponding to a slow and controlled addition of the glycidol monomer and optionally of its co-monomers to the reaction medium.

The macropolyol can also be obtained by co-polymerization with other functionalized monomers which can incorporate at least one group selected from fluorinated, silane, siloxane and halogenated compounds such as propylene oxide, ethylene oxide, butylene oxide, epichlorohydrin, vinyloxirane, glycidyl allyl ether, glycidyl methacrylate, isopropyl glycidyl ether, phenyl glycidyl ether, 2-ethylhexyl glycidyl ether, hexadecyl glycidyl ether, naphthyl glycidyl ether, t-butyldimethylsilyl-(R)-(−)-glycidyl ether, benzyl glycidyl ether, epoxy-3-phenoxypropane, biphenyl glycidyl ether, propargyl glycidyl ether, n-alkyl glycidyl ethers, but also functionalized oxiranes such as γ-glycidylpropyltrimethoxysilane, γ-glycidylpropyltriethoxysilane γ-glycidoxypropyl-bis(trimethylsiloxy)-methylsilane and 3-(bis(trimethylsiloxy)methyl)-propyl glycidyl ether, glycidylglycerol ether, glycidyl butylether, glycidylnonylphenylether, fluorinated and perfluorinated oxiranes such as hexafluoropropylene oxide, 2,3-difluoro-2,3-bis-trifluoromethyl-oxirane, 2,2,3-trifluoro-3-pentafluoroethyl-oxirane, 2,3-difluoro-2-(1,2,2,2-tetrafluoro-1-trifluoromethyl-ethyl)-3-trifluoromethyl-oxirane, 2-fluoro-2-pentafluoroethyl-3,3-bis-trifluoromethyl-oxirane, 1,2,2,3,3,4,4,5,5,6-decafluoro-7-oxa-bicyclo[4.1.0]heptane, 2,3-difluoro-2-trifluoromethyl-3-pentafluoroethyl-oxirane, 2,3-difluoro-2-nonafluorobutyl-3-trifluoromethyl-oxirane, 2,3-difluoro-2-heptafluoropropyl-3-pentafluoroethyl-oxirane, 2-fluoro-3-pentafluoroethyl-2,3-bis-trifluoromethyl-oxirane, 2,3-bis-pentafluoroethyl-2,3-bistrifluoromethyl-oxirane, 2,3-bis-trifluoromethyl-oxirane, 2-pentafluoroethyl-3-trifluoromethyl-oxirane, 2-(1,2,2,2-tetrafluoro-1-trifluoromethyl-ethyl)-3-trifluoromethyl-oxirane, 2-nonafluorobutyl-3-pentafluoroethyl-oxirane, 2,2-bis-trifluoromethyl-oxirane 2-heptafluoroisopropyloxirane, 2-heptafluoropropyloxirane, 2-nonafluorobutyloxirane, 2-tridecafluorohexyloxirane, and HFP (hexafluoropropene) trimer oxiranes including 2-pentafluoroethyl-2-(1,2,2,2-tetrafluoro-1-trifluoromethyl-ethyl)-3,3-bis-trifluoromethyl-oxirane, 2-fluoro-3,3-bis-(1,2,2,2-tetrafluoro-1-trifluoromethyl-ethyl)-2-trifluoromethyl-oxirane, 2-fluoro-3-heptafluoropropyl-2-(1,2,2,2-tetrafluoro-1-trifluoromethyl-ethyl)-3-trifluoromethyl-oxirane and 2-(1,2,2,3,3,3-hexafluoro-1-trifluoromethyl-propyl)-2,3,3-tris-trifluoromethyl-oxirane. These types of compounds or moieties can be introduced in situ by copolymerization or by post-modification.

The crosslinkable composition may comprise, in addition to the polyol used in the preparation of the prepolymer, optionally at least one polyol, for example 1, or 2, or 3, or 4 polyols, wherein the polyol may preferably comprise at least two hydroxyl groups. Advantageously, the polyol may serve as a chain extender or co-crosslinker.

The polyol may be of the di- or multifunctional poly(ethylene glycol) (PEG) or poly(propylene glycol) (PPG) type, depending on the type of initiator used. In particular, it can be prepared by ring-opening polymerization of ethylene oxide and/or propylene oxide from a polyol or a monofunctional alcohol selected from ethylene glycol, diethylene glycol, triethylene glycol, trimethylolpropane, glycerol, pentaerythritol, xylitol, sorbitol, ethanol, butanol, phenol, 1,2-propylene glycol, dipropylene glycol, 1,4-butane diol and hexamethylene glycol.

The monofunctional polyols or alcohols may alternatively be molecular polyols of the type ethylene glycol, diethylene glycol, triethylene glycol, trimethylolpropane, glycerol, pentaerythritol, xylitol, sorbitol, ethanol, butanol, phenol, 1,2-propylene glycol, dipropylene glycol, polypropylene glycol, 1,4-butane diol, or hexamethylene glycol. The polyol can be obtained by any method known to the skilled person, for example by using one or more mono- or polyfunctional initiators such as those indicated above for the macropolyol.

The polyol can alternatively be a diol incorporating silane or siloxane groups such as hydroxyl-terminated dihydroxytelechelic polydimethylsiloxane, but also diblock or triblock block copolymers of polydimethylsiloxane and polycaprolactone, of polydimethylsiloxane and polyoxyethylene or of polydimethylsiloxane and polypropylene glycol.

The polyols used can alternatively be linear or branched copolymers, for example based on PEG/PPG such as Pluronic® or Tetronic®. Advantageously, the copolymers can be as described in the document EP2496620 ([1]).

The composition of the invention may optionally comprise at least one monofunctional alcohol, a mixture of monofunctional alcohols or a monohydroxylated polyether based on ethylene glycol and/or ethylene glycol/propylene glycol, or a mixture of monohydroxylated polyether and monofunctional alcohols. It may be at least one, for example 2, or 3, or 4, monohydroxy polyethers or monofunctional alcohols, preferably 1 or 2. “Ethylene glycol and/or ethylene glycol/propylene glycol based” means a polyether essentially consisting of ethylene glycol monomer units. Advantageously, they can be used to limit and/or control the crosslinking rate, and thus control the density of the network, and consequently all the properties ranging from mechanical properties to maximum water contents, etc. They can be included in the composition of the invention in an amount of 0 to 20% by weight.

The composition of the invention may optionally comprise at least one catalyst or combination of catalysts. Advantageously, the catalyst may be capable of catalyzing the polymerization or accelerating the reaction rate. The catalyst may be selected from bismuth, tin, titanium or organobismuth based organometallics, for example bismuth(III) tricarboxylates, bismuth-2-ethylhexanoate, bismuth neodecanoate, tin dibutyl laurate, tin octoate (Sn(oct)₂), and/or an orthotitanate such as tetra-n-butylorthotitanate, iron tetrachloride, tertiary amines such as triethylamine, tributylamine or any other commonly used trialkylamine as well as nucleophilic molecules such as 1,4-diazabicyclo[2.2.2]octane (DABCO), triazabicyclodecene (TBD), 1,5-diazabicyclo[4.3.0]non-5-ene (DBN) or 1,8-diazabicyclo[5.4.0]undec-7-ene (DBU).

The composition of the invention may optionally comprise at least one additive for example selected from:
antioxidants, such as BHA (butyl hydroxyl anisole), BHT (butylated hydroxytoluene) or ascorbic acid, beta-carotene, glutathione, lipoic acid, retinol, santowhite, uric acid, ubiquinol, vitamin E. An antioxidant may be present, in the composition of the invention, between 0.02 and 2% by weight, for example between 0.05 and 1% by weight, preferentially 0.05 and 0.5% by weight of the reactants.
oxygen permeability promoters, such as fluorinated and silylated co-monomers used in the synthesis of polyglycerol, or fluorinated or silylated polyols used during hydrogel formation,
plasticizers or viscosity modifiers of the formulation before crosslinking, such as glycols or polyols miscible with polyglycerol and allowing to reduce its viscosity,
humectants or water retention agents, for example poly(ethylene glycol) dimethyl ether (PEGDME),
lubricants, such as hyaluronic acid or PEG
compatibilizing agents, which advantageously ensure a good homogeneity of the formulation,
coloring agents and/or optical brighteners. These may be any agents known to the person skilled in the art for this purpose, such as for example those described in the US Code of Federal Regulations (Color Additives Listed for Use in Medical Devices: Exempt from Certification (21 CFR 73, Subpart D); Color Additives Listed for Use in Medical Devices: Subject to Certification (21 CFR 74, Subpart D ([4])), and more particularly 1,4-bis[(2-hydroxyethyl)amino]-9,10-anthracenedione bis(2-methyl-2-propenoic)ester copolymers, 1,4-bis[(2-methylphenyl)amino]-9,10-anthracenedione, 1,4-bis[4-(2-methacryloxyethyl) phenylamino]anthraquinone copolymers, carbazole violet, soluble oil of chlorophyll-copper complex, chromium-cobalt-aluminum oxide, chromium oxide green, C.I. Vat Orange 1 (Colour Index No. 59105), 2-[[2,5-diethoxy-4-[(4-methylphenyl)thiol]phenyl]azo]-1,3,5-benzenetriol, 16,23-dihydrodinaphtho[2,3-a:2′,3′-i] naphth [2′,3′:6,7] indolo [2,3-c] carbazole-5,10,15,17,22,24-hexone, N,N′-(9,10-dihydro-9,10-dioxo-1,5-anthracenediyl) bisbenzamide, 7,16-dichloro-6,15-dihydro-5,9,14,18-anthrazinetrone, 16,17-dimethoxydinaphtho [1,2,3-cd:3′,2′,1′-lm] perylene-5,10-dione, poly(hydroxyethyl methacrylate) and dye copolymers, 4-[(2,4-dimethylphenyl)azo]-2,4-dihydro-5-methyl-2-phenyl-3H-pyrazol-3-one, 6-ethoxy-2-(6-ethoxy-3-oxobenzothien-2(3H)-ylidene) benzo[b]thiophen-3 (2H)-one, phthalocyanine green, iron oxides, titanium dioxide, reaction products of vinyl alcohol/methyl methacrylate and a dye, mica-based pearlescent pigments, disodium 1-amino-4-[[4-[(2-bromo-1-oxoallyl)amino]-2-sulfonatophenyl]amino]-9,10-dihydro-9,10-dioxoanthracene-2-sulfonate. The agents may also be those authorized to tint medical devices according to the legal provisions in force in Europe, Japan, Korea, this list not being restrictive. The coloring agents can be for example incorporated into the hydrogel in the post-polymerization hydration step.
opacifying agents, such as titanium dioxide. At least one opacifying agent can be used, at a concentration comprised between 0.0001% and 0.08% by weight of the reagents, for example 0.05%.
anti-microbial agents,
anti-biofilm agents,
therapeutic agents,
modulus modifiers. These can be, for example, linear polymers not reactive with isocyanates, such as polyvinylpyrrolidone or polyethylene glycol dialkyl ethers (PEGDME), which are incorporated into the polyurethane crosslinked network, leading to semi-interpenetrating networks (semi-IPNs).

The at least one additive may represent between 0 and 20% of the total mass of the composition of the invention. For example, it may be between 1 and 18%, or between 2 and 15%, or between 5 and 10% of the total mass of the composition of the invention.

When the crosslinked or crosslinkable composition comprises an agent selected from UV filters, UV absorbers and blue-light filters, it may be, for example, any commercially available UV filter, such as AEHB (acryloxyethoxyhydroxybenzophenone), and/or any UV absorber having high absorption in the UV-A range (320-380 nm) but relatively transparent above 380 nm. Generally, if the UV absorber is present in the composition of the invention, it is present between 0.5 and 1.5% by weight of the reactants, for example at 1% by weight. The UV absorbers can be incorporated into the hydrogel at the post-polymerization hydration step.

Advantageously, the crosslinkable or crosslinked composition of the invention may comprise, as a percentage by weight based on the total weight of the composition:
from 5-80% macropolyol (5-80%), for example from 10 to 70%, or from 20 to 50%,
from 0-50% polyol, for example from 1 to 45%, or from 5 to 40%,
from 1-60% diisocyanate, triisocyanate or polyisocyanate, for example from 1 to 50%, or from 5 to 40%,
from 0-60% polyurethane prepolymer, for example from 5 to 50%, or from 10 to 50%, or from 20 to 40%,
   formulated so as to obtain a film, which in hydrated medium provides a hydrogel, having the advantageous characteristics as defined above.

Advantageously, the crosslinked composition of the invention comprises:
at least one di- or polyisocyanate and at least one polyglycerol, in particular a hyperbranched polyglycerol as defined above, or
at least one multifunctional isocyanate-terminated urethane prepolymer as defined above and at least one polyglycerol, in particular a hyperbranched polyglycerol as defined above, or
at least one diisocyanate, at least one oligoglycerol and at least one hyperbranched polyglycerol, or
at least one diisocyanate, at least one multifunctional isocyanate-terminated urethane prepolymer comprising from 1 to 16 isocyanate functionalities on average, the average functionality being strictly greater than 1, produced by the reaction of a diisocyanate with a monofunctional polyol or alcohol comprising 1 to 8 hydroxyl groups, at least one oligoglycerol and at least one hyperbranched polyglycerol, or
at least one diisocyanate, at least one multifunctional isocyanate-terminated urethane prepolymer comprising from 1 to 16 isocyanate functionalities on average, produced by the reaction of a diisocyanate with a polyol comprising 1 to 8 hydroxyl groups, and at least one hyperbranched polyglycerol or
at least one diisocyanate, at least one multifunctional isocyanate-terminated urethane prepolymer also comprising from 1 to 16 isocyanate functionalities on average (the average functionality being strictly greater than 1), produced by the reaction of a diisocyanate with a polyol comprising 1 to 8 hydroxyl groups, at least one polyol, at least one hyperbranched polyglycerol and optionally at least one oligoglycerol, or
at least one multifunctional isocyanate-terminated urethane prepolymer also comprising from 1 to 16 isocyanate functionalities on average (the average functionality being strictly greater than 1), produced by the reaction of a diisocyanate with a polyol comprising 1 to 8 hydroxyl groups, at least one polyol and at least one hyperbranched polyglycerol.

Another object of the invention relates to a hydrogel obtainable by water absorption/water swelling of a crosslinked composition according to the invention, or, when an aprotic solvent is present in the crosslinked composition, by exchange of the aprotic solvent with excess water.

Hydrogels contain, after hydration, a certain amount of water, which can be determined by means of successive mass measurements after hydration (for example after 12 h, with distilled water or solutions having the physicochemical characteristics of tear fluid) and after drying (for example for 12 h, at 90° C.). The water content (EWC, in %) can be expressed according to equation 1 below:

$$EWC = \frac{w - {w}_{0}}{{w}_{0}} \times 100 \qquad ( 1 )$$

With w representing the mass in the hydrated state, and wo the mass in the “dehydrated” or dry state.
Similarly, a swelling ratio (SR, in %) can be determined according to equation 2 below

$$SR = \frac{w - {w}_{0}}{w} \times 100 \qquad ( 2 )$$

Advantageously, the hydrogel has a theoretical permeability allowing the applications mentioned below. According to the previously determined water content and according to the equation of Fatt & Chaston (3), a theoretical permeability (Dk) in Barrer of the hydrogel can be estimated:

Dk=2.0×10⁻¹¹xe^{0.0411 EWC} (3)

The mechanical properties of the hydrogel, including Young's modulus, stress at break, and elongation at break, can be determined by any method known to the skilled person, for example, by means of a tensile machine. Preferably, the tested samples are all of similar dimensions (for example, 3 mm wide by 1 cm long), and the thickness can be measured with each new analysis. The analysis can be started with a preload of 0.1 N at a strain rate equivalent to 20 mm/min.

The surface properties of the designed materials can also be analyzed via various techniques known to the skilled person, such as optical microscopy, atomic force microscopy, wettability, coefficient of friction and surface roughness measurements.

Another object of the invention relates to a process for preparing a crosslinked composition according to the invention, consisting of reacting, in the presence or absence of aprotic solvent, preferably in the absence of solvent:

A1) at least one urethane prepolymer as defined above, with a polyol or a monofunctional alcohol comprising 1 to 8, preferably 2 to 3, hydroxyl groups; and/or
A2) at least one di- or polyisocyanate and/or oligoglycerol;
with
B) at least one macropolyol as defined above;
and:
C) optionally at least one polyol as defined above;
D) optionally at least one monoalcohol, a mixture of monoalcohols or a monohydric polyether based on ethylene glycol and/or ethylene glycol/propylene glycol, or a mixture of monohydric polyether and monoalcohols as defined above;
E) optionally at least one catalyst or combination of catalysts as defined above;
F) optionally at least one additive as defined above; and
G) optionally at least one agent selected from UV filters, UV absorbers and blue-light filters as defined above.

The aprotic solvent, when used, is selected from polar aprotic solvents such as dimethylformamide, acetonitrile, dimethylacetamide and dimethylsulfoxide, or a mixture of at least two of these.

The process of the invention can be carried out under ambient conditions and/or under anhydrous conditions and under inert atmosphere. In the sense of the present invention, “atmosphere and ambient conditions” means non-anhydrous conditions, a non-inert atmosphere, at about 25° C. (±3° C.). The ingredients must be liquid either because of a higher temperature if necessary, obtained for example by heating, or because of the presence of solvent, or because of the use of low molar mass compounds. These low molar mass compounds can be diisocyanates which can liquefy solid prepolymers, and/or polyols with a degree of polymerization less than or equal to 7, for example glycols to liquefy potentially solid high molar mass polyols and macropolyols. Therefore, the temperature of the process of the invention can be comprised between 10° C. and 90° C., preferentially between 25° C. and 80° C.

The duration of the process of the invention can be such as to allow the total consumption of the isocyanate functions, either by reaction with the hydroxyl functions of the polyols and macropolyols when the reaction is carried out in anhydrous medium, or with the hydroxyl functions of the polyols, macropolyols and water if the reaction is carried out in the presence of water in limited and controlled amounts.

The various components used in the context of the process of the invention can be produced by means known to the skilled person, for example by mixing with a spatula, using a mechanical stirrer, or any mixers used by the skilled person for multi-component thermosetting formulations, such as, for example, with a speed mixer, with a two-component static mixer or using ultrasound.

Advantageously, the reaction mixture is relatively anhydrous, i.e., it contains little or no water, and in any case not as reactants. Indeed, to avoid an increase in modulus, which is undesirable in the case of materials used for the lens industry, the absence of urea groups or bonds in the gel composition is an advantage.

Alternatively, the reaction mixture may contain a controlled amount of water. Advantageously, this embodiment can control certain properties, such as improved water retention or modulus. At the end of the mixing step, the resulting mixture can be reacted under an inert atmosphere to form a three-dimensional crosslinked polyurethane network, which can be molded by various methods known to the skilled person. Advantageously, the step of molding the composition during the crosslinking can provide the composition with the desired shape. These methods can be, for example, spin-casting, or cast-molding. The molding step can take place under an inert atmosphere, in particular under an oxygen/nitrogen atmosphere, or under a controlled humidity atmosphere, i.e., either under an inert and therefore dry atmosphere, or under controlled humidity leading to the controlled formation of urea functions. This step can be carried out, for example, at a temperature comprised between 10 and 140° C., preferably between room temperature (i.e., about 25° C.) and 90° C., preferentially between 40 and 80° C.

The films, once in the molds, are placed in an oven at 40 to 100° C., preferably at 50 to 80° C. for 1 to 20 h, preferably 4 to 10 h, for example 8 h.

An annealing can also be applied, between 60 and 150° C., preferably between 70 and 100° C., for 2 min to 2 h, for example 20 min to 1 h. The characterization of the network by IR analysis can confirm the complete disappearance of isocyanate groups in the medium.

The process may further comprise a step of hydrating/swelling the crosslinked composition with excess water, to form a hydrogel. Alternatively, when an aprotic solvent is used, the process may further comprise a step of exchanging the aprotic solvent with excess water, advantageously to replace the aprotic solvent used in the preparation of the mixtures.

The process may further comprise a step selected from molding, lathe cutting, casting, two-component mixing, and speed mixing.

Another object of the invention relates to an article obtainable by a process for preparing a crosslinked composition according to the invention as defined above. The article may be a medical device, such as a contact or intraocular lens, a patch, a dressing or a medical implant for tissue engineering and/or delivery of active ingredients, or a superabsorbent material.

Another object of the invention relates to the use of a composition or hydrogel according to the invention for the manufacture of a medical device, such as a contact or intraocular lens, a patch, a dressing or a medical implant for tissue engineering and/or delivery of active ingredients, or a superabsorbent material.

Another object of the invention relates to the use of a composition or hydrogel according to the invention as a carrier for a compound of interest selected from therapeutic active ingredients, vitamins, nutrients, decontaminating agents or lubricants.

Among the active therapeutic ingredients or “therapeutic agents”, mention may be made of anti-inflammatory drugs, for example non-steroidal anti-inflammatory drugs (NSAIDs) or corticoids, antibiotics, alone or in combination, anti-glaucoma drugs, alone or in combination, anti-allergic drugs, alone or in combination, drugs for the treatment of the progression of myopia such as, for example, anticholinergic drugs, drugs for the treatment of presbyopia, and, more generally, treatments of ocular pathologies including the eye in its entirety and its appendages: eyelids, oculomotor muscle, lacrimal glands and their secretions, and orbits.

Non-drug substances may include vitamins and nutrients such as antioxidants, protective agents for the metabolism of the eye and its appendages, and lens decontaminants such as bacterial anti-biofilms, antifungals, antiamebics and antivirals.

The substances can be introduced after polymerization or before.

Another object of the invention relates to the use of a crosslinked composition as defined above, capable of forming a hydrogel polymer by swelling in the presence of a necessary and sufficient amount of water, for the manufacture of a medical device, such as a contact or intraocular lens, a patch, a dressing or a medical implant for tissue engineering and/or delivery of active ingredients and/or surgery. The crosslinked composition also has applications in the so-called “domestic” fields, for example as a perfume diffuser, in cosmetics, in the field of diapers, and in fields relating to the preservation of the environment, as for example with depollutants.

For example, the crosslinked composition can be derived from the reaction in the presence or absence of an aprotic solvent, under anhydrous conditions and under inert atmosphere, or under ambient atmosphere and conditions:

(a) of at least either an oligoglycerol, a dendrimer, an optionally functionalized linear, branched or hyperbranched polyglycerol, comprising at least 8 hydroxyl groups with at least one di- or polyisocyanate; optionally in the presence of at least one catalyst and/or optionally at least one additive selected from antioxidants, oxygen permeability promoters, plasticizers, humectants, lubricants, viscosity modifiers, compatibilizing agents, coloring agents, opacifying agents, antimicrobial agents, modulus modifiers, therapeutic agents and bacterial anti-biofilm agents; and/or optionally at least one agent selected from UV filters, UV absorbers and blue-light filters; or

(b) at least either an oligoglycerol, a dendrimer, a linear, branched or hyperbranched polyglycerol, optionally functionalized, comprising at least 10 hydroxyl groups, with at least one di- or polyisocyanate, and at least one polyol comprising 2 to 6, preferably 2 to 3, hydroxyl groups; optionally in the presence of at least one catalyst and/or optionally at least one additive selected from antioxidants, modulus modifiers, oxygen permeability modulators, plasticizers, humectants, lubricants, viscosity modifiers, compatibilizing agents, coloring agents, opacifying agents, antimicrobial agents, therapeutic agents, and bacterial anti-biofilm agents; and/or optionally at least one agent selected from UV filters, UV absorbers, and blue-light filters.

Other advantages may become apparent to the skilled person upon reading the examples below.

### EXAMPLES

The acronyms PEG200, PEG300, PEG400 and PEG600 stand for poly(ethylene glycol) compounds having an average molar mass of 200, 300, 400 and 600 g·mol⁻¹.
The acronym HPG stands for hyperbranched macropolyol
HDI stands for hexamethylene diisocyanate
h-MDI stands for 4,4′-bismethylene(cycloisocyanate)
IPDI stands for isophorone diisocyanate
The notation TMP refers to trimethylolpropane
MeOK refers to potassium methanolate
The acronym PDMS500 is used to represent the dihydroxylated telechelic polydimethylsiloxane of average molar mass 500 g·mol⁻¹
MeO(PEGx)OH represents a heterotelechelic α-methoxy-ω-hydroxy poly(ethylene glycol) of average molar mass x g·mol⁻¹
DBTDL refers to the metal catalyst tin dibutyl dilaurate
DMF refers to the organic solvent N,N-dimethylformamide
The acronym PRP is used to refer to prepolymers
The notation PRP ADI-PEGx describes the composition of the synthesized prepolymer with ADI coding for the nature of the aliphatic diisocyanate used and PEGx coding for the PEG used and x its average molar mass
TMPEO is used to designate polyol trimethylolpropane ethoxylate. Its composition is specified via the notation xEO/yOH
DG is used to designate the tetrafunctional polyol diglycerol

### Determination of the Properties Implemented in the Examples

Determination of Oxygen Permeability (Dk) by Polarography

The polarographic method is based on a classical electrochemical setup with 3 electrodes: gold working electrode (WE), platinum counter electrode (CE) and Ag/AgCl reference electrode (RE), immersed in a 0.1 M electrolyte solution (KCl). The hydrogel is placed on the surface of the working electrode, then oxygen is injected into the electrochemical cell and the current variation is measured (oxidation of the oxygen on the surface of the WE). The intensity of the current measured will depend on the amount of oxygen passed through the hydrogel.

Three tests are performed for each sample, and the average of the three analyses is retained.

The potentiostat used for these analyses is a DropSens pSTAT 400.

### Determination of Mechanical Properties

Using a tensile testing machine (M500-30AT) equipped with a DBBMTCL 50 kg test cell, the mechanical properties determined are: Young's modulus, stress at break and elongation at break.

The dimensions of the hydrated samples are normalized to 3 mm width for 10 mm between the jaws, the thickness is measured at each new analysis. The preload is 0.1 N for a deformation rate equivalent to 20 mm/min all at room temperature. All specimens were evaluated at least 3 times and averages of the data calculated by the WinTest software were calculated.

### Determination of Surface Properties

The designed materials are also analyzed via different techniques including wettability measurements (a), surface roughness by atomic force microscopy (AFM) (b), friction coefficients by tribometer (c)

(a) Wettability Measurements

Surface properties were also determined by measurements of water contact angles on the materials using the drop-on-solid mode on a KRUSS DSA 100 apparatus. Briefly, a drop (2 μL) of distilled water is deposited on the material surface and the angle (in°) at equilibrium of the drop with the material is measured via a video camera. An average of 10 measurements is taken via DropShapeAnalysis software.

(b) Surface Roughness Measurements by Atomic Force Microscopy (AFM)

AFM analyses were performed on a Bruker Dimension EDGE instrument. The analyses were performed in Tapping mode. Levers of force 3 N/m with Si3N4 tips (Bruker, Product code: RFESP) were used to generate the phase, amplitude and height images. The height images allowed us to access the R(max), Ra and Rq, defined respectively as the maximum height identified on the sample surface; the average surface roughness and the standard deviation of the average flat surface. Samples were scanned at lengths of 20, 10, 5 and 1 μm to generate scanned areas of 400, 100, 25 and 1 μm². Data were processed with Nanoscope Analysis software and compared with commercial lenses.

(c) Measurement of Friction Coefficients with a Tribometer

The measurements were performed on a CSM instrument tribometer. A steel ball of 10 mm diameter was used at a speed of 1 cm/sec, with a normal force of 0.5 N. 3 analyses were performed on each sample and an average of the 3 was calculated. The sample is in film form, and the analysis takes place in liquid medium (water or saline) at room temperature.

### UV Transmittance Analyses

Transmittance was determined using a UV spectrophotometer. A lens is placed in a cell containing a saline solution. The cell is placed in the sample compartment. A cell containing only saline solution is placed in the reference compartment.

And the spectrum in % transmittance is recorded between 200 and 780 nm. The sample is analyzed 3 times and an average of the 3 measurements at 550 nm was retained.

### Water Content and Swelling Rate

The water content and swelling rate are determined by measuring the weight of the gel in the dry and hydrated state using Equations 1 and 2.

The gels in the hydrated state are weighed individually after removing excess water from the surface. The gels are then dried in an oven at 80° C. for a minimum of 6 h and weighed again. This process is repeated 3 times, and the EWC value is the average of the 3.

### Example 1: Synthesis of Macropolyol of Mₙ(Theoretical)=6000 g·mol⁻¹

In a 250 mL three-necked flask, trimethylolpropane (TMP; 1 eq, 1.17 g, 8.7×10⁻³ mol) and 25% potassium methanolate in methanol (MeOK; 0.3 eq, 0.72 g, 2.6×10⁻³ mol) are introduced.

The flask is then placed in the rotary evaporator at 70° C. until complete dissolution of the TMP, then the rotary evaporator is put under vacuum to remove the methanol.

The three-necked flask containing the reagents is then placed in an oil bath thermostated at 95° C., topped by a stirring paddle (300 rpm) under nitrogen flow. Once the reaction medium is at temperature, glycidol (80 eq, 50 g, 0.675 mol) is added with a peristaltic addition pump at a rate of 3.6 m L/h.

Once the addition of glycidol is completed, the reaction medium is left under stirring for a few hours before being stopped. The obtained polymer is dissolved in methanol, deionized with Amberlite® and then precipitated twice in acetone.

The obtained macropolyol is characterized by size-exclusion chromatography (SEC) and ¹H NMR spectroscopy

δ (ppm), MeOD: 4.92 (OH); 3.56 (CH₂—CH₂—O)ₙ₋₂; 1.36 (CH₂, TMP); 0.87 (CH₃, TMP)

### Example 2: Synthesis of Macropolyol of Mₙ(Theoretical)=4000 g·mol⁻¹

The same protocol as that of Example 1 was used to produce a macropolyol with an average molar mass of 4000 g·mol⁻¹ by changing the proportions of the reactants according to Table 1.

### Example 3: Synthesis of Macropolyol of Mₙ(Theoretical)=2000 g·mol⁻¹

The same protocol as that of Example 1 was used to produce a macropolyol with an average molar mass of 2000 g·mol⁻¹ by changing the proportions of the reactants according to Table 1.

### Example 4: Synthesis of Macropolyol of Mₙ(Theoretical)=1000 g·mol⁻¹

The same protocol as that of Example 1 was used to produce a macropolyol with an average molar mass of 1000 g·mol⁻¹ by changing the proportions of the reactants according to Table 1.

TABLE 1
Conditions for the synthesis of macropolyols

| Macropolyol | TMP | MeOK | glycidol |
|---|---|---|---|
| synthesis | (g) | (g) | (g) |
| Example 1: | 1.17 | 0.72 | 50 |
| 6000 g · mol⁻¹ | | | |
| Example 2: | 1.77 | 0.843 | 50 |
| 4000 g · mol⁻¹ | | | |
| Example 3: | 3.62 | 2.27 | 50 |
| 2000 g · mol⁻¹ | | | |
| Example 4: | 6.04 | 3.79 | 40 |
| 1000 g · mol⁻¹ | | | |

### Example 5: General Process for Preparing Solventless Gels Based on Macropolyol and Aliphatic Diisocyanates

Macropolyol is introduced with one or more aliphatic diisocyanates into a suitable vial. The vial is closed and introduced into a Speed Mixer for 3 min at 2500 rpm. The vial is then placed in a thermostatic oven for 2 to 24 h depending on the nature of the isocyanates used. The compositions of the gel formulations are detailed in Table 2. Amounts are expressed in percentages by weight. The water contents (EWC) are given in Table 3.

TABLE 2
Composition of solventless gels based on
macropolyol and aliphatic diisocyanates

| | %(wt) | %(wt) | %(wt) | | | |
|---|---|---|---|---|---|---|
| | HPG1000 | HPG4000 | HPG₂₀₀₀ | %(wt) | %(wt) | %(wt) |
| Ex | ex. 4 | ex. 2 | ex. 3 | IPDI | HDI | h-MDI |
| A1 | | 82 | | 18 | | |
| A2 | 70.6 | | | 29.4 | | |
| A3 | | 90.4 | | | | 9.6 |
| A4 | | 85.6 | | | | 14.4 |
| A5 | 74.8 | | | | | 25.2 |
| A6 | 66.5 | | | | 33.5 | |
| A7 | 43.5 | | | | | 56.5 |
| A8 | | | 74.8 | | 25.2 | |
| A9 | | | 65.6 | | | 34.4 |
| A10 | 54.3 | | | | 45.7 | |
| A11 | | | 64.3 | 35.7 | | |
| A12 | | 81 | | | 19 | |

TABLE 3
Water content of hydrogels

| | A8 | A12 |
|---|---|---|
| EWC(%)^{a} | 78.2 | 72.4 |
| SR(%)^{b} | 359 | 262.3 |

^{a}EWC water content determined by Equation 1;
^{b}SR swelling rate determined by Equation 2

### Example 6: General Process for Preparing Solvent-Free Gels Based on Macropolyol, Aliphatic Diisocyanates and Polyols

The macropolyol and polyol are introduced with one or more aliphatic diisocyanates into a suitable vial. The vial is closed and introduced into a Speed Mixer for 3 min at 2500 rpm. The vial is then placed in a thermostatic oven for 2 to 24 h at 80° C. depending on the nature of the isocyanates used. The compositions of the gel formulations are detailed in Table 4. Amounts are expressed in percentages by weight. The water content is given in Table 5.

TABLE 4
Composition of solventless gels based on macropolyol, polyols and aliphatic diisocyanates

| | %(wt) | %(wt) | %(wt) | | | | | | | |
|---|---|---|---|---|---|---|---|---|---|---|
| | HPG₁₀₀₀ | HPG₄₀₀₀ | HPG₂₀₀₀ | %(wt) | %(wt) | %(wt) | %(wt) | %(wt) | %(wt) | %(wt) |
| Ex. | ex. 4 | ex. 2 | ex. 3 | IPDI | HDI | h-MDI | TMPEO_{(20EO/3OH)} | PEG₄₀₀ | PEG₆₀₀ | DG |
| B1 | | 58.4 | | 12.7 | | | 28.9 | | | |
| B2 | 39.9 | | | 18.5 | | | 41.6 | | | |
| B3 | | 57.4 | | | | 14.2 | 28.4 | | | |
| B4 | 40.9 | | | | | 17.7 | 41.4 | | | |
| B5 | 34.8 | | | | 23.4 | | | 41.8 | | |
| B6 | | | 47.5 | | 24 | | | | 28.5 | |
| B7 | 34.8 | | | | 23.4 | | | | 41.8 | |
| B8 | | | 42.1 | 32.7 | | | | 25.2 | | |
| B9 | | 59.9 | | | 22.9 | | 17.2 | | | |
| B10 | | 58 | | | 22.2 | | | 19.8 | | |
| B11 | | | 41.9 | | | 33 | | | 25.1 | |
| B12 | 37.2 | | | | 25 | | 37.8 | | | |
| B13 | 37.9 | | | | | 49.5 | | | | 12.6 |
| B14 | 46 | | | | 38.7 | | | | | 15.3 |

TABLE 5
Water content of hydrogels

| | B5 | B6 | B7 | B8 | B9 | B12 |
|---|---|---|---|---|---|---|
| EWC(%)^{a} | 42.6 | 48.4 | 67.3 | 59.4 | 62.5 | 57.8 |
| SR(%)^{b} | 74.2 | 93.8 | 205.3 | 146.6 | 166.9 | 137 |

^{b}EWC water content determined by Equation 1;
^{b}SR swelling rate determined by Equation 2

### Example 7: General Process for Preparing Solvent-Based Gels Based on Macropolyol, Aliphatic Diisocyanates with or without Polyols

The macropolyol (and polyol if needed) are introduced into a vial, named A, with 75% of the total solvent volume, and homogenized in a Speed Mixer for 2 min at 2500 rpm. A second vial, named B, is then prepared by mixing one or more aliphatic diisocyanates with 25% of total DMF. The vial is closed in turn and introduced into the Speed Mixer for 1 min at 2500 rpm. The contents of B are poured into A and homogenized. The closed vial is left for 5 min at room temperature before being placed in a thermostatic oven at 80° C. for 2 to 24 h depending on the nature of the isocyanates used. The compositions of the gel formulations are detailed in Table 4. The amounts of reagents are expressed in mass percentages without taking into account the solvent. The amount of solvent used is such that it generally represents 75 to 80 wt % of the total formulation, which is equivalent to having a 75/25 solvent/total reagents ratio. The water content of some of these gels is shown in Table 6.

TABLE 6
Composition of gels prepared with solvent from macropolyol, polyols and aliphatic diisocyanates

| | %(wt) | %(wt) | | | | | | | | |
|---|---|---|---|---|---|---|---|---|---|---|
| | HPG₄₀₀₀ | HPG₂₀₀₀ | %(wt) | %(wt) | %(wt) | %(wt) | %(wt) | %(wt) | % wt total | [DMF] |
| | ex. 2 | ex. 3 | IPDI | HDI | h-MDI | TMPEO_{(20EO/3OH)} | PEG₂₀₀ | PEG₆₀₀ | reagents | (% wt) |
| C1 | 54.7 | | 28.7 | | | 16.7 | | | 12.9 | 87.1 |
| C2 | 52.3 | | | 21 | | 26.7 | | | 16.7 | 83.3 |
| C3 | 76.1 | | 19.3 | | | | 4.5 | | 20.6 | 79.4 |
| C4 | 79.2 | | | 15.4 | | | 5.4 | | 21.6 | 78.4 |
| C5 | 79.7 | | | | 20.2 | | 3.1 | | 25 | 75 |
| C6 | | 59.9 | 24.9 | | | 15.2 | | | 25.9 | 74.1 |
| C7 | | 59.8 | 24.9 | | | | | 15.3 | 26 | 74 |
| C8 | | 60.1 | | 24.7 | | 15.2 | | | 26.2 | 73.8 |
| C9 | | 60 | | 24.7 | | | | 15.3 | 25.9 | 74.1 |
| C10 | | 56.2 | | | 29.5 | 14.3 | | | 23 | 77 |

TABLE 7
Composition of gels with solvent based on macropolyol,
polyols and aliphatic diisocyanates

| | C3 | C4 | C5 | C8 |
|---|---|---|---|---|
| EWC(%)^{a} | 65.2 | 68.1 | 58 | 64.7 |
| SR(%)^{b} | 187.5 | 217 | 138.1 | 183 |

^{a}EWC water content determined by Equation 1;
^{b}SR swelling rate determined by Equation 2

### Example 8: Formation of a PEG₃₀₀-IPDI Prepolymer

Poly(ethylene glycol) (M_{w}=300 g/mol) is previously dried under vacuum at 90° C. for 24 h before use. PEG (4.993 g, 17 mmol) is introduced into a 250 mL three-necked flask, topped with a semi-circular stirring paddle. The device is placed under an inert atmosphere and placed in an oil bath thermostated at 50° C., under stirring (200 rpm).

The diisocyanate, in this case isophorone diisocyanate (IPDI) (7.4 g, 7.1 mL, 33 mmol) is taken under nitrogen flow and added to the reaction medium. The reaction is thus left for 2 h until complete functionalization of the OH groups at the end of the PEG chain into urethane function. The product is characterized by ¹H NMR, CES and IR and then stored in a sealed vial at 5° C.

δ(ppm), CDCl₃: δ 4.96+4.70 (s, 2H, NH.; 4.18 (m, NH(CO)O—CH₂); 3.62 (m, (CH₂—CH₂—O)ₙ; 2.92 (m, CH—NCO and/or CH₂—NCO); 1.77+0.92 (m, CH ring) 0.99 (s, CH₃ *3)

### Example 9: Formation of a PEG₃₀₀-HDI Prepolymer

The same protocol was used to produce a prepolymer between PEG₃₀₀ and hexamethylene diisocyanate by changing the proportions of the reactants according to Table 6.

δ (ppm), CDCl₃: δ 4.93 (s, 2H, NH); 4.22 (s, CH₂—O—(CO)); 3.66 (m, (CH₂—CH₂—O)ₙ); 3.31 (t, CH₂—NCO); 3.16 (t, CH₂—NH—(CO)); 1.62+1.52+1.43 (m, CH₂—(CH₂)₂—CH₂).

### Example 10: Formation of a PEG₃₀₀-h-MDI Prepolymer

The same protocol was used to produce a prepolymer between PEG₃₀₀ and 4.4′-methylenebis(cycloisocyanate) by changing the proportions of the reactants according to Table 6.

δ (ppm), CDCl₃: 4.93 (s, NH); 4.22 (s, CH₂—O—(CO)); 3.66 (m, (CH₂—CH₂—O)); 3.31 (t, CH₂—NCO); 3.16 (t, CH₂—NH—(CO)); 1.62+1.52+1.43 (m, CH₂—(CH₂)₂—CH₂).

### Example 11: Formation of a PEG₂₀₀-IPDI Prepolymer

The same protocol was used to produce a prepolymer between PEG₂₀₀ and isophorone diisocyanate by changing the proportions of the reactants according to Table 6.

δ (ppm), CDCl₃: δ 4.96+4.70 (s, 2H, NH.; 4.18 (m, NH(CO)O—CH₂); 3.62 (m, (CH₂—CH₂—O)ₙ; 2.92 (m, CH—NCO and/or CH₂—NCO); 1.77+0.92 (m, CH cycle) 0.99 (s, CH₃ *3)

### Example 12: Formation of a PEG₂₀₀-HDI Prepolymer

The same protocol was used to produce a prepolymer between PEG₂₀₀ and hexamethylene diisocyanate by changing the proportions of the reactants according to Table 6.

δ (ppm), CDCl₃: δ 4.93 (s, 2H, NH); 4.22 (s, CH₂—O—(CO)); 3.66 (m, (CH₂—CH₂—O)ₙ); 3.31 (t, CH₂—NCO); 3.16 (t, CH₂—NH—(CO)); 1.62+1.52+1.43 (m, CH₂—(CH₂)₂—CH₂).

### Example 13: Formation of a PEG₂₀₀-h-MDI Prepolymer

The same protocol was used to produce a prepolymer between PEG₂₀₀ and 4.4′-methylenebis(cycloisocyanate) by changing the proportions of the reactants according to Table 8.

δ (ppm), CDCl₃: 4.93 (s, NH); 4.22 (s, CH₂—O—(CO)); 3.66 (m, (CH₂—CH₂—O)); 3.31 (t, CH₂—NCO); 3.16 (t, CH₂—NH—(CO)); 1.62+1.52+1.43 (m, CH₂—(CH₂)₂—CH₂).

TABLE 8
Synthesis conditions of prepolymers

| | | PEG₃₀₀ | HDI | h-MDI | IPDI | PEG₂₀₀ |
|---|---|---|---|---|---|---|
| | | (g) | (mL) | (mL) | (mL) | (g) |
| Ex. 8 | IPDI-PEG₃₀₀ | 5 | | | 7.1 | |
| Ex. 9 | HDI-PEG₃₀₀ | 5 | | 5.3 | | |
| Ex. 10 | h-MDI-PEG₃₀₀ | 5 | 8.4 | | | |
| Ex. 11 | IPDI-PEG₂₀₀ | | | | 10.5 | 5 |
| Ex. 12 | HDI-PEG₂₀₀ | | 8 | | | 5 |
| Ex. 13 | h-MDI-PEG₂₀₀ | | | 12.3 | | 5 |

### Example 14: General Process for Preparing Solventless Gels Based on Macropolyol of Aliphatic Urethane Diisocyanate Prepolymers and Optionally Aliphatic Diisocyanates

The macropolyol is introduced with one or more aliphatic urethane diisocyanate prepolymers and optionally with an aliphatic diisocyanate. The vial is closed and introduced into the Speed Mixer for 3 min at 2500 rpm. The vial is then placed in a thermostatic oven for 2 to 24 h depending on the nature of the isocyanates used. The compositions of the gel formulations are detailed in Table 9. Amounts are expressed in percent by weight. The water content of some of these gels is shown in Table 10.

TABLE 9
Composition of gels from macropolyol and urethane prepolymer diisocyanates
and optionally aliphatic diisocyanates without solvent

| | | | | %(wt) | %(wt) | %(wt) | %(wt) | %(wt) | |
|---|---|---|---|---|---|---|---|---|---|
| | %(wt) | %(wt) | %(wt) | HDI- | h-MDI- | IPDI- | h-MDI- | IPDI- | |
| | HPG₁₀₀₀ | HPG₂₀₀₀ | HPG₄₀₀₀ | PEG₂₀₀ | PEG₂₀₀ | PEG₂₀₀ | PEG₃₀₀ | PEG₃₀₀ | %(wt) |
| | ex. 4 | ex. 3 | ex. 2 | ex. 12 | ex. 13 | ex. 11 | ex. 10 | ex. 8 | HDI |
| D1 | | | 67 | 33 | | | | | |
| D2 | | | 59.2 | 40.8 | | | | | |
| D3 | 35.9 | | | | 64.1 | | | | |
| D4 | 24.7 | | | | | | 75.3 | | |
| D5 | 21.3 | | | 34 | | 33.9 | | | 10.8 |
| D6 | 22 | | | | 78 | | | | |
| D7 | | 51 | | | | 49 | | | |
| D8 | | 43 | | 57 | | | | | |
| D9 | | 37.7 | | | | | 62.3 | | |
| D10 | | | 56.4 | | | | | 43.6 | |
| D11 | 60 | | | | | | | 40 | |
| D12 | 21.3 | | | 22.6 | 45.3 | | | | 10.8 |
| D13 | 18.7 | | | 30.2 | | | | 41.6 | 9.5 |

TABLE 10
Water content (EWC) of gels

| | D5 | D8 | D10 | D11 | D12 |
|---|---|---|---|---|---|
| EWC(%)^{a} | 51.9 | 24.6 | 42.2 | 24.6 | 35.5 |
| SR(%)^{b} | 111.5 | 32.6 | 73.1 | 32 | 112 |

^{a}EWC water content determined by Equation 1;
^{b}SR swelling rate determined by Equation 2

### Example 15: General Process for Preparing Solvent-Free Gels Based on Macropolyol, Aliphatic Urethane Diisocyanate Prepolymers, Optionally Aliphatic Diisocyanates and Polyols

The macropolyol and polyol are introduced with one or more aliphatic urethane diisocyanate prepolymers and optionally with an aliphatic diisocyanate. The vial is closed and introduced into the Speed Mixer for 3 min at 2500 rpm. The vial is then placed in a thermostatic oven for 2 to 24 h at 80° C. depending on the nature of the isocyanates used. The compositions of the gel formulations are detailed in Table 11. Amounts are expressed in percent by weight. The water content (EWC) is given in Table 12.

TABLE 11
Composition of gels from macropolyol, polyols, urethane prepolymer diisocyanates
and optionally aliphatic diisocyanates without solvent

| | | | %(wt) | %(wt) | %(wt) | %(wt) | | | |
|---|---|---|---|---|---|---|---|---|---|
| | %(wt) | %(wt) | HDI- | HDI- | IPDI- | h-MDI- | | | |
| | HPG₁₀₀₀ | HPG₄₀₀₀ | PEG₂₀₀ | PEG₃₀₀ | PEG₂₀₀ | PEG₃₀₀ | %(wt) | %(wt) | %(wt) |
| | ex. 4 | ex. 2 | ex. 12 | ex. 9 | ex. 11 | ex. 10 | TMPEO_{(20EO/3OH)} | PEG₂₀₀ | HDI |
| E1 | | 40.1 | | | 38.7 | | 21.1 | | |
| E2 | 18 | | 29 | | 34 | | | 11 | 9 |
| E3 | 22.6 | | | | 45.9 | | 31.5 | | |
| E4 | | 32 | | | | 41 | | 16 | 10 |
| E5 | 18 | | 28 | 34 | | | | 11 | 9 |

TABLE 12
Water content (EWC) of hydrogels

| | E1 | E2 | E3 | E5 |
|---|---|---|---|---|
| EWC(%)^{a} | 26.8 | 35.88 | 21.1 | 57.58 |
| SR(%)^{b} | 36.8 | 55.95 | 26.8 | 135.71 |

(c) EWC water content determined by Equation 1;
^{b}SR swelling rate determined by Equation 2

### Example 16: Process for Preparing Gels with Solvent Based on Macropolyol, Aliphatic Urethane Diisocyanate Prepolymers with or without Polyols

The macropolyol (and polyol if needed) are introduced into a vial, named A, with 75% of the total solvent volume, and homogenized in a Speed Mixer for 2 min at 2500 rpm. A second vial, named B, is then prepared by mixing one or more aliphatic diisocyanates with 25% of total DMF. The vial is closed in turn and introduced into the Speed Mixer for 1 min at 2500 rpm. The contents of B are poured into A and homogenized. The closed vial is left for 5 min at room temperature before being placed in a thermostatic oven at 80° C. for 2 to 24 h depending on the nature of the isocyanates used. The compositions of the gel formulations are detailed in Table 13. The amounts of reagents are expressed in percentage by weight, without taking into account the solvent. The amount of solvent used is such that it generally represents 55 to 85 wt % of the total formulation. The water contents of these gels after hydration are listed in Table 14.

TABLE 13
Composition of gels from macropolyol, polyols, urethane diisocyanate prepolymer with solvent

| | | %(wt) | %(wt) | %(wt) | %(wt) | %(wt) | | | | | | |
|---|---|---|---|---|---|---|---|---|---|---|---|---|
| | %(wt) | HDI- | h-MDI- | IPDI- | HDI- | h-MDI- | | | | | %(wt) | |
| | HPG₄₀₀₀ | PEG₃₀₀ | PEG₃₀₀ | PEG₂₀₀ | PEG₂₀₀ | PEG₂₀₀ | %(wt) | %(wt) | %(wt) | %(wt) | PDMS | %(wt) |
| | ex. 2 | ex. 9 | ex. 10 | ex. 11 | ex. 12 | ex. 13 | TMPEO_{(20EO/3OH)} | PEG₄₀₀ | PEG₆₀₀ | DG | 500 | DMF |
| F1 | 59 | | | | 25 | | 16 | | | | | 75 |
| F2 | 41.7 | 37.6 | | | | | | | 20.7 | | | 75 |
| F3 | 51.3 | | | | | 41.3 | | 7.4 | | | | 75 |
| F4 | 53 | | | 39.4 | | | 7.6 | | | | | 75 |
| F5 | 59 | | | | 41 | | | | | | | 85 |
| F6 | 34.2 | | | | 16.5 | 37.2 | | | 13.6 | | | 72 |
| F7 | 36.5 | 31.4 | 25.1 | | | | | 7 | | | | 75 |
| F8 | 31 | 16.5 | 49.6 | | | | | | | 3 | | 75 |
| F9 | 34.5 | 30.4 | 24.1 | | | | | | | | 11 | 75 |
| F10 | 53.7 | | | | 32 | | 14.3 | | | | | 75 |
| F11 | 58.2 | | | | 39.3 | | 2.5 | | | | | 75 |
| F12 | 32.7 | | 41.2 | | 18.2 | | | | | | 7.9 | 75 |
| F13 | 49.7 | | | | 50.3 | | | | | | | 75 |
| F14 | 32.7 | 30.1 | | | 20.5 | | | | | | 17 | 75 |
| F15 | 49.8 | 50.2 | | | | | | | | | | 75 |

TABLE 14
Composition of gels with solvent based on macropolyol,
polyols and aliphatic urethane diisocyanate prepolymers

| | F1 | F2 | F3 | F4 | F5 | F6 | F7 | F8 |
|---|---|---|---|---|---|---|---|---|
| EWC(%)^{a} | 86.7 | 71.3 | 56.7 | 86.1 | 67.1 | 72.1 | 61.5 | 67.1 |
| SR(%)^{b} | 654.3 | 248.5 | 131.1 | 623.6 | 204.2 | 258.5 | 159.7 | 204.2 |

| | F9 | F10 | F11 | F12 | F13 | F14 | F15 |
|---|---|---|---|---|---|---|---|
| EWC(%)^{a} | 67.5 | 83.8 | 70.6 | 55.6 | 48.8 | 65.6 | 60.1 |
| SR(%)^{b} | 208 | 516.9 | 240 | 125 | 109.7 | 190.4 | 135.1 |

(d) EWC water content determined by Equation 1;
^{b}SR swelling rate determined by Equation 2

### Example 17: Shaping and Studying the Properties of Gels

The properties of the compositions detailed in Tables 8 and 9 are listed in Table 15.

TABLE 15
Physical properties of hydrogels (transmission
TR and mechanical properties)

| | TR- | E | σ | ε |
|---|---|---|---|---|
| | UV(%)^{a} | (MPa)^{b} | (MPa)^{c} | (%)^{d} |
| F2 | 95 | 1.1 | 0.6 | 43 |
| F3 | 97 | — | — | — |
| F6 | 95 | 0.9 | 0.8 | 92.5 |
| F7 | 94 | 0.2 | 0.3 | 90 |
| F8 | 97 | 0.2 | 0.3 | 94 |
| F9 | 97 | 0.2 | 0.3 | 132 |
| F11 | — | 1.1 | 0.7 | 53.7 |

^{a}determined by UV-VIS transmission spectroscopy at 550 nm;
^{b}Young's modulus, average of at least 3 analyses;
^{c}stress at break, average of at least 3 analyses;
^{d}elongation at break; average of at least 3 analyses

### Example 18: Specific Properties of Gels: Surface Roughness

The formulated and hydrated gels were also studied by AFM spectroscopy to determine the surface roughness. The properties of one of the formed gels are listed in Table 16.

TABLE 16
Surface roughness properties of a hydrogel

| | R₍ₘₐₓ₎ | | |
|---|---|---|---|
| | (nm)^{a} | Rₐ(nm)^{b} | R_{q}(nm)^{c} |
| F10 | 38 | 2.95 | 3.26 |

^{a}total profile height: height between the deepest valley and the highest peak over the evaluation length,
^{b}arithmetic mean profile roughness: defined over a base length,
^{c}root mean square profile roughness: corresponds to the standard deviation of the height distribution

### Example 19: Specific Properties of Gels: Wettability

The formulated and hydrated gels were also studied for wettability to determine the contact angle between the formulated lens and a drop of water. The contact angles obtained for some of these gels are listed in Table 17 and are the result of an average over 10 analyses.

TABLE 17
Surface properties of a hydrogel in wettability (water angle in °)

| | F10 | F5 | F11 |
|---|---|---|---|
| θ | 63.2 | 54.7 | 34.1 |

### Example 20: Specific Properties of Gels: Water Retention

Dehydration kinetics of the formulated and hydrated gels in parallel with dehydration kinetics of commercial lenses were performed. The percentages of water remaining in the gels at times of 20, 40, and 60 minutes are listed in Table 18.

TABLE 18
Water retention properties (in %)

| | 20 min | 40 min | 60 min |
|---|---|---|---|
| SI—H | 46 | 22 | 15 |
| HR55 | 28 | 10 | 2 |
| HEMA ref | 86 | 75 | 66 |
| F8 | 88 | 78 | 70 |
| F9 | 74 | 56 | 46 |

Si—H: silicone lens
HR55: PHEMA commercial lens
HEMA Ref: HEMA gel

TABLE 19
Water retention properties, comparison of time required
for 10% water loss (t_{10%}) and for 50% water loss (t_{50%})

| Sample name | t_{10%} (min)^{a} | t_{50%} (min)^{b} |
|---|---|---|
| Si—H | 3.41 | 20.61 |
| HR55 | 3.07 | 15.92 |
| HEMA Ref | 2.75 | 17.06 |
| F8 | 9.81 | 39.53 |
| F9 | 10.72 | 64.4 |

^{a}time necessary for the loss of 10% of water initially present in the network;
^{b}time necessary for the loss of 50% of water initially present in the network
Si—H: silicone lens
HR55: PHEMA commercial lens
HEMA Ref: HEMA gel

### Example 21: Specific Properties of Gels: Oxygen Permeability

According to the protocol listed above, the oxygen permeability of hydrogels and commercial references was determined and is presented in Table 20.

TABLE 20
Oxygen permeability properties, comparison
with commercial formulations

| | Ref | Ref | | | |
|---|---|---|---|---|---|
| | HEMA | HR55 | F12 | F14 | F13 |
| EWC (%) | 44 | 55 | 55.6 | 65.6 | 48.8 |
| e (cm) | 0.071 | 0.024 | 0.11 | 0.061 | 0.1015 |
| D × 10⁵(cm²/s) | 2.03 | | 3.46 | 3.47 | 1.7 |
| k × 10⁴ | 6.69 | | 9.30 | 7.91 | 12.51 |
| (mLO₂/(cm³ · cmHg)) | | | | | |
| Dk^{a} | 13.6 | 13.4 | 32.2 | 27.4 | 21.7 |
| Dk/e^{b} | 19.2 | 55.9 | 29.3 | 44.9 | 21.4 |

^{a}With Dk expressed in Barrer or in 10⁻¹⁰ ((mL O₂ · cm)/(s · cm²/cmHg))
^{b}With Dk/e expressed in 10⁻⁹ ((mL O₂))/(s · cm²/cmHg))

### REFERENCES

1. EP2496620.
2. Nomenclature and Terminology for Dendrimers with Regular Dendrons and for Hyperbranched Polymers A. Fradet, J. Kahovec, IUPAC Nomenclature Project Nr: 2001-081-1-800.
3. J. Am. Chem. Soc. 2000, 122, 2954-2955.
4. 21 CFR 73, Subpart D); Color Additives Listed for Use in Medical Devices: Subject to Certification (21 CFR 74, Subpart D.

1. A crosslinkable composition comprising:
A1) at least one multifunctional isocyanate-terminated urethane prepolymer comprising from 1 to 16 isocyanate functionalities on average, the average functionality being strictly greater than 1, said prepolymer being a product of the reaction of a diisocyanate, a triisocyanate or a polyisocyanate of functionality strictly greater than 3, with a polyol or a monofunctional alcohol comprising 1 to 8 hydroxyl groups; and/or
A2) at least one mono-, di- or polyisocyanate and/or oligoglycerol;
with
B) at least one macropolyol selected from:
B1) oligoglycerols with an average degree of polymerization less than or equal to 7,
B2) glycerol dendrimers,
B3) linear, branched or hyperbranched polyglycerols with a degree of polymerization greater than or equal to 8,
B4) mixtures of hyperbranched polyglycerols and linear, branched or hyperbranched oligoglycerols, with a degree of polymerization comprised between 2 and 7, optionally functionalized;
and optionally:
C) at least one polyol comprising at least two hydroxyl groups;
D) at least one mono-, di- or polyisocyanate;
E) optionally at least one monofunctional alcohol, a mixture of monofunctional alcohols or a monohydroxy polyether based on ethylene glycol and/or ethylene glycol/propylene glycol, or a mixture of monohydroxy polyether and monofunctional alcohols;
F) at least one catalyst or combination of catalysts;
G) at least one additive selected from antioxidants, oxygen permeability promoters, water retention agents, lubricants, compatibilizing agents, viscosity modifying agents, coloring agents, opacifying agents, antimicrobial agents, therapeutic agents, and bacterial anti-biofilm agents; and/or
H) at least one agent selected from UV filters, UV absorbers and blue-light filters.

2. A crosslinked composition, capable of forming a hydrogel polymer by absorption of water, resulting from the crosslinking of a crosslinkable composition as claimed in claim 1, either under anhydrous conditions and under inert atmosphere, or under ambient atmosphere.

3. The composition as claimed in claim 1, comprising:
at least one di- or polyisocyanate and at least one polyglycerol, in particular a hyperbranched polyglycerol, or
at least one multifunctional isocyanate-terminated urethane prepolymer as defined in claim 1 and at least one polyglycerol, in particular a hyperbranched polyglycerol, or
at least one diisocyanate, at least one oligoglycerol and at least one hyperbranched polyglycerol, or
at least one diisocyanate, at least one multifunctional isocyanate-terminated urethane prepolymer as defined in claim 1, at least one oligoglycerol and at least one hyperbranched polyglycerol, or
at least one diisocyanate, at least one multifunctional isocyanate-terminated urethane prepolymer as defined in claim 1, and at least one hyperbranched polyglycerol or
at least one diisocyanate, at least one multifunctional isocyanate-terminated urethane prepolymer as defined in claim 1, at least one polyol, at least one hyperbranched polyglycerol and optionally at least one oligoglycerol, or
at least one multifunctional isocyanate-terminated urethane prepolymer as defined in claim 1, at least one polyol and at least one hyperbranched polyglycerol.

4. The composition as claimed in claim 1, wherein the multifunctional urethane isocyanate prepolymer is derived:
from a diisocyanate OCN—R₁—NCO, wherein R₁ represents a linear or branched, monocyclic or polycyclic or acyclic C₁ to C₁₅ alkylene, a C₁ to C₄ alkylidene, or a C₆-C₁₀ arylene optionally bearing at least one substituent selected from linear or branched C₁-C₆ alkyl, C₁-C₂ alkylene, or a halogen atom; or
from a polyisocyanate with a functionality greater than 2.

5. The composition as claimed in claim 4, wherein the multifunctional isocyanate-terminated urethane prepolymer is derived:
from a diisocyanate selected from methylene dicyclohexyl diisocyanate, hexamethylene diisocyanate, isophorone diisocyanate, toluene-2,4-diisocyanate, toluene-2,6-diisocyanate, mixtures of toluene-2,4 and 2,6-diisocyanates, ethylene diisocyanate, ethylidene diisocyanate, propylene-1,2-diisocyanate, cyclohexylene-1,2-diisocyanate, cyclohexylene-1,4-diisocyanate, m-phenylene diisocyanate, 4,4′-biphenylene diisocyanate, 3,3′-dichloro-4,4′-biphenylene diisocyanate, 1,4-tetramethylene diisocyanate, 1,10-decamethylene diisocyanate, cumene-2,4 diisocyanate, 1,5-naphthalene diisocyanate, 1,4-cyclohexylene diisocyanate, 2,5-fluorenediisocyanate, 2-2′-diphenylmethylene diisocyanate, 4,4′-diphenylmethylene diisocyanate, 4,4′-dibenzyl diisocyanate, m-xylylene diisocyanate, hexamethylene diisocyanate trimer and polymers of 4,4′-diphenylmethane diisocyanate, diphenyl-4,4″-biphenylene diisocyanate, 1,6-hexamethylene diisocyanate, m-phenylene diisocyanate, polymers of 4,4′-diphenylmethane diisocyanate, p-tetramethyl xylylene diisocyanate, p-phenylene diisocyanate, 4-methoxy-1,3-phenylene diisocyanate, 4-chloro-1,3-phenylene diisocyanate, 4-bromo-1,3-phenylene diisocyanate, 4-ethoxy-1,3-phenylene diisocyanate, 2,4-dimethyl-1,3-phenylene diisocyanate, 5,6-dimethyl-1,3-phenylene diisocyanate, 2,4-diisocyanatodiphenylether, 4,4″-diisocyanatodiphenylether, benzidine diisocyanate, 4,6-dimethyl-1,3-phenylene diisocyanate, 14-anthracene diisocyanate, 4,4′-diisocyanatodibenzyl, 3,3′-dimethyl-4,4′-diisocyanatodiphenylmethane, 2,6-dimethyl-4,4′-diisocyanatodibenzyl, 2,4-diisocyanatostilbene, 3,3′-dimethoxy-4,4′-diisocyanatodiphenyl, 2,5-fluorenediisocyanate, 1,8-naphthalene diisocyanate, 2,6-diisocyanatobenzofuran, xylene diisocyanate, m-tetramethyl xylylene diisocyanate or methylene diisocyanatodiphenyl; preferably hexamethylene diisocyanate, isophorone diisocyanate and methylenebis(4-cyclohexyl)diisocyanate; or
from a polyisocyanate with a functionality greater than 2, for example the trifunctional trimer (isocyanurate) of isophorone diisocyanate, or the trifunctional trimer (isocyanurate) of hexamethylene diisocyanate and 4.4-diphenyl methane diisocyanate polymer, as well as the corresponding allophanates, biurets or uretdiones.

6. The composition as claimed in claim 1, wherein the multifunctional isocyanate-terminated urethane prepolymer is derived from a polyol selected from linear or branched poly(ethylene glycols) (PEG) or poly(propylene glycols) (PPG) comprising at least one hydroxyl function; molecular polyols comprising at least one hydroxyl function; co-polymers of poly(ethylene glycols) (PEG) and or poly(propylene glycols) (PPG); and diols comprising silanes or polysiloxanes with terminal hydroxyl functions.

7. The composition as claimed in claim 6, wherein the multifunctional isocyanate-terminated urethane prepolymer is derived from a polyol or monofunctional alcohol selected from ethylene glycol, diethylene glycol, triethylene glycol, trimethylolpropane, glycerol, pentaerythritol, xylitol, sorbitol, ethanol, butanol, phenol, 1,2-propylene glycol, dipropylene glycol, 1,4-butane diol, hexamethylene glycol, polyethylene glycol, polypropylene glycol, polydimethylsiloxane, linear or branched PEG/PPG copolymers.

8. A hydrogel obtainable by means of a crosslinked composition as claimed in claim 2, by absorption of water/by swelling with water, or, when an aprotic solvent is present in said crosslinked composition, by exchange of the aprotic solvent with excess water.

9. A process for preparing a crosslinked composition as claimed in claim 2 comprising reacting, in the presence or absence of aprotic solvent, preferably in the absence of solvent:
A1) at least one multifunctional isocyanate-terminated urethane prepolymer comprising from 1 to 16 isocyanate functionalities on average, the average functionality being strictly greater than 1, said prepolymer being a product of the reaction of a diisocyanate, a triisocyanate or a polyisocyanate of functionality strictly greater than 3, with a polyol or a monofunctional alcohol comprising 1 to 8 hydroxyl groups; and/or
A2) at least one mono-, di- or polyisocyanate and/or oligoglycerol;
with
B) at least one macropolyol selected from:
B1) oligoglycerols with an average degree of polymerization less than or equal to 7,
B2) polyglycerol dendrimers,
B3) linear, branched or hyperbranched polyglycerols with a degree of polymerization greater than or equal to 8,
B4) mixtures of hyperbranched polyglycerols and linear, branched or hyperbranched oligoglycerols, with a degree of polymerization comprised between 2 and 7, optionally functionalized;
optionally in the presence of:
C) at least one polyol comprising at least two hydroxyl groups;
D) at least one mono-, di- or polyisocyanate;
E) optionally at least one monofunctional alcohol, a mixture of monofunctional alcohols or a monohydroxy polyether based on ethylene glycol and/or ethylene glycol/propylene glycol, or a mixture of monohydroxy polyether and monofunctional alcohols;
F) at least one catalyst or combination of catalysts;
G) at least one additive selected from antioxidants, oxygen permeability promoters, water retention agents, lubricants, compatibilizing agents, coloring agents, viscosity modifying agents, opacifying agents, antimicrobial agents, therapeutic agents and bacterial anti-biofilm agents; and/or
H) at least one agent selected from UV filters, UV absorbers and blue-light filters.

10. The process as claimed in claim 9, wherein:
the catalyst, when used, is selected from bismuth, tin or titanium organobismuth based organometallics, for example bismuth(III) tricarboxylate, bismuth-2-ethylhexanoate, bismuth neodecanoate, tin dibutyl laurate, tin octoate, and/or orthotitanate, iron tetrachloride, and tertiary amines such as triethylamine, and/or
the aprotic solvent, when used, is selected from polar aprotic solvents such as dimethylformamide, acetonitrile, dimethylacetamide and dimethylsulfoxide, or a mixture of at least two of these.

11. The process as claimed in claim 9, further comprising a step selected from molding, lathe cutting, casting, two-component mixing, and speed mixing.

12. The process as claimed in claim 9, further comprising a step of molding the composition upon curing.

13. The process as claimed in claim 9, further comprising a step of hydrating/swelling the crosslinked composition with excess water, or, when an aprotic solvent is used, a step of exchanging the aprotic solvent with excess water.

14. An article obtainable by a process as claimed in claim 9 or comprising a hydrogel as defined by absorption of water, resulting from the crosslinking of a crosslinkable composition by absorption of water/by swelling with water, or, when an aprotic solvent is present in said crosslinked composition, by exchange of the aprotic solvent with excess water.

15. The article of claim 14, which is a medical device, such as a contact or intraocular lens, a patch, a dressing or a medical implant for tissue engineering and/or delivery of active ingredients, or a superabsorbent material.

16. The use of a crosslinked composition as defined in claim 2 or a hydrogel as defined by absorption of water, resulting from the crosslinking of a crosslinkable composition by absorption of water/by swelling with water, or, when an aprotic solvent is present in said crosslinked composition, by exchange of the aprotic solvent with excess water for the manufacture of a medical device, such as a contact or intraocular lens, a patch, a dressing or a medical implant for tissue engineering and/or delivery of active ingredients, or a superabsorbent material.

17. The use of a crosslinked composition as defined in claim 2 or a hydrogel as defined by absorption of water, resulting from the crosslinking of a crosslinkable composition by absorption of water/by swelling with water, or, when an aprotic solvent is present in said crosslinked composition, by exchange of the aprotic solvent with excess water as a carrier for a compound of interest selected from therapeutic active ingredients, vitamins, nutrients, decontaminating agents or lubricants.

18. The use of a crosslinked composition capable of forming a hydrogel polymer by swelling in the presence of an excess of water, for the manufacture of a medical device, such as a contact or intraocular lens, a patch, a dressing or a medical implant for tissue engineering and/or delivery of active ingredients; the crosslinked composition being derived from the reaction in the presence or absence of an aprotic solvent, under anhydrous conditions and under inert atmosphere, or under ambient atmosphere and conditions:
1. of at least either an oligoglycerol, a dendrimer, an optionally functionalized linear, branched or hyperbranched polyglycerol, comprising at least 8 hydroxyl groups with at least one di- or polyisocyanate; optionally in the presence of at least one catalyst and/or optionally at least one additive selected from antioxidants, oxygen permeability promoters, plasticizers, humectants, modulus modifiers, lubricants, viscosity modifiers, compatibilizing agents, coloring agents, opacifying agents, antimicrobial agents, therapeutic agents and bacterial anti-biofilm agents; and/or optionally at least one agent selected from UV filters, UV absorbers, and blue-light filters; or
2. of at least either an oligoglycerol, a dendrimer, a linear, branched or hyperbranched polyglycerol, optionally functionalized, comprising at least 10 hydroxyl groups, with at least one di- or polyisocyanate, and at least one polyol comprising 2 to 6, preferably 2 to 3, hydroxyl groups; optionally in the presence of at least one catalyst and/or optionally at least one additive selected from antioxidants, modulus modifiers, oxygen permeability modulators, plasticizers, humectants, lubricants, viscosity modifiers, compatibilizing agents, coloring agents, opacifying agents, antimicrobial agents, therapeutic agents, and bacterial anti-biofilm agents; and/or optionally at least one agent selected from UV filters, UV absorbers, and blue-light filters.
